(19) United States
(12) Patent Application Publication
(10) Pub. No.: US 2024/0279591 A1
(43) Pub. Date: Aug. 22, 2024

(54) COMPOSITIONS COMPRISING SPHERICAL PARTICLES AND USES OF SAME

(71) Applicants: Nano Ghost Ltd, Netanya (IL); Technion Research & Development Foundation Limited, Haifa (IL)

(72) Inventors: Osnat BOHANA-KASHTAN, Tel-Mond (IL); Oded PINKAS, Petach-Tikva (IL); Irina GOTLIV, Rehovot (IL); Haim TSUBERY, Elad (IL); Tal Yoetz KOPELMAN, Tel Aviv (IL); Neta CHAIM, Degania Alef (IL); Noa AVNI, Haifa (IL); Hanna LIANDRES, Tel Aviv (IL); Roy GOVRIN, Kiryat Tivon (IL); Yonatan MALCA, Tel Aviv (IL); Marcelle MACHLUF, Haifa (IL); Orit AMSALEM-BERGELSON, Rehovot (IL)

(73) Assignees: Nano Ghost Ltd, Netanya (IL); Technion Research & Development Foundation Limited, Haifa (IL)

(21) Appl. No.: 18/569,642

(22) PCT Filed: Jun. 12, 2022
(86) PCT No.: PCT/IL2022/050627

§ 371 Date: Dec. 13, 2023

Related U.S. Application Data
(60) Provisional application No. 63/210,032, filed on Jun. 13, 2021.

(51) Int. Cl. C12M 1/00; A61K 9/48; C12N 5/00

(52) U.S. Cl. CPC C12M 47/06; A61K 9/48; C12N 5/0068; C12N 2521/00

(57) ABSTRACT

A method of producing spherical particles is provided herein. Also provided are compositions generated thereby and uses thereof in disease diagnosis and treatment.

### RELATED APPLICATION/S

This application claims the benefit of priority of U.S. Provisional Patent Application No. 63/210,032 filed on 13 Jun. 2021, the contents of which are incorporated herein by reference in their entirety.

### FIELD AND BACKGROUND OF THE INVENTION

The present invention, in some embodiments thereof, relates to compositions comprising spherical particles and uses of same.

Targeted cancer therapy is the ultimate goal of the pharmaceutical industry. However, most platforms have relatively short circulation times and highly complicated production methods that hinder their clinical applications.

WO2011/024172 disclose a targeted delivery platform, based on nanoghosts (NGs) that are generated from the whole cell membrane of mesenchymal stem cells (MSCs). NGs derived from MSCs comprise typical MSC markers which ensure their targeting to cancer cells. In contrast to exosomes or other extracellular vesicles that are shed or bud from cells and their size is small (30-150 nm), MSC-NGs are manufactured in a reproducible controlled process by isolating intact MSC cell membranes (ghost cells), and homogenizing them into nanosized vesicles (nanoghosts) while entrapping a therapeutic of choice. This approach, has become a platform for active cancer-targeted drug delivery as it is biocompatible, characterized by long duration time within target site, and selectivity.

### RELATED ART

Boone, C. W., Ford, L. E., Bond, H. E., Stuart, D. C. & Lorenz, D. Isolation of plasma membrane fragments from HeLa cells. J Cell Biol 41, 378-392 (1969).
Westerman and Jensen Methods Enzymol. 2003; 373:118-27.
Busatto, et al. Tangential flow filtration for highly efficient concentration of extracellular vesicles from large volumes of fluid. Cells 12: 273 (2018)

### SUMMARY OF THE INVENTION

According to an aspect of some embodiments of the present invention there is provided a method of producing spherical particles, the method comprising:
(a) subjecting cells to a hypotonic treatment so as to obtain swollen intact cells;
(b) subjecting the swollen intact cells to flow shearing to obtain ruptured cells while avoiding nuclei lysis;
(c) filtering the ruptured cells to obtain a cell preparation devoid of nuclei;
(d) subjecting the cell preparation devoid of nuclei to size separation to obtain ghosts;
(e) downsizing the ghosts using a high shear homogenizer or microfluidizer, to obtain particles of 35-400 nm; and optionally
(f) purifying the particles to obtain the spherical particles.

According to some embodiments of the invention, the cells are mesenchymal stem cells (MSCs).

According to some embodiments of the invention, the MSCs are cultured in suspension.

According to some embodiments of the invention, the cells are provided at an amount of at least 0.5×10⁹.

According to some embodiments of the invention, the MSCs exhibit a population doubling level (PDL) of 15-30.

According to some embodiments of the invention, the method further comprises culturing the cells prior to step (a).

According to some embodiments of the invention, the culturing is in a bioreactor.

According to some embodiments of the invention, the cells are a pure population of cells being ≥95% MSCs.

According to some embodiments of the invention, the hypotonic treatment is under dynamic conditions.

According to some embodiments of the invention, osmolarity of the hypotonic treatment is 5-100 mOsm/Kg.

According to some embodiments of the invention, the hypotonic treatment is effected for 10-60 minutes.

According to some embodiments of the invention, the hypotonic treatment results in cell swelling by at least 20% as determined by cell diameter.

According to some embodiments of the invention, the flow shearing is performed by a needle, tube or channel.

According to some embodiments of the invention, the flow shearing is performed with a single or multi-needle/tube/channel apparatus.

According to some embodiments of the invention, the flow shearing is done with a needle/tube/channel characterized by an internal diameter (ID) of 100-400 μm and/or a needle/tube/channel length of 2-50 mm.

According to some embodiments of the invention, the filtering the ruptured cells to obtain a cell preparation devoid of nuclei comprises a first filtration at a filter cut-off of 1.2-10 μm to remove nuclei, and optionally a second filtration at a filter cut-off of 0.45-0.85 μm to remove intracellular organelles.

According to some embodiments of the invention, the size separation is performed by a size exclusion column.

According to some embodiments of the invention, the size separation is performed by Tangential Flow Filtration (TFF).

According to some embodiments of the invention, operating conditions for the high shear homogenizer or microfluidizer include at least 100 bar.

According to some embodiments of the invention, the high shear homogenizer is of 100-2000 bar

According to some embodiments of the invention, the method further comprises subjecting the ghosts to size separation following the downsizing.

According to some embodiments of the invention, the purifying comprises filtering the particles to obtain spherical particles at a filter cut-off of 0.2-0.22 μm.

According to an aspect of some embodiments of the present invention there is provided a composition comprising a plurality of spherical particles composed of a whole cell membrane fraction, wherein the spherical particles exhibit native membrane symmetry and expression of native markers obtainable according to the method as described herein.

According to some embodiments of the invention, the composition is characterized by a membrane to nucleus marker ratio higher than that obtained when using a homogenizer instead of the flow shearing to rupture cells or when collecting the centrifugal pellet following homogenization instead of the filtrate following the flow shearing

According to an aspect of some embodiments of the present invention there is provided a method of producing a pharmaceutical composition, the method comprising producing the composition as described herein and adding a pharmaceutical agent of interest following the size separation and prior to the downsizing so as to obtain spherical particles encapsulating the pharmaceutical agent.

According to an aspect of some embodiments of the present invention there is provided a composition comprising a plurality of spherical particles as described herein According to an aspect of some embodiments of the present invention there is provided a pharmaceutical composition comprising the composition as described herein and a pharmaceutically acceptable carrier or diluent.

According to an aspect of some embodiments of the present invention there is provided a pharmaceutical composition as described herein for use in treating cancer.

According to some embodiments of the invention, the pharmaceutical agent comprises a water insoluble drug.

According to some embodiments of the invention, the water insoluble drug comprises irinotecan or its active derivative SN-38.

According to some embodiments of the invention, the pharmaceutical agent comprises an mRNA.

According to some embodiments of the invention, the mRNA encodes IL-12.

According to some embodiments of the invention, the water insoluble drug is in a formulation for increasing water solubility.

According to some embodiments of the invention, the formulation comprises the water insoluble drug attached to a cyclodextrin, a protein or a peptide.

According to an aspect of some embodiments of the present invention there is provided a composition—of matter comprising a plurality of spherical particles composed of a whole cell membrane fraction, wherein the spherical particles exhibit native membrane symmetry and expression of native markers, the spherical particles encapsulating or conjugated to a pharmaceutical agent being exogenous to the cell from which the whole cell membrane is obtained, wherein the pharmaceutical agent is selected from the group consisting of an immune activating chemokine or cytokine, Poly (ADP-ribose) polymerase (PARP) inhibitor (PARPi), a topoisomerase 1 inhibitor and an immunogenic peptide.

According to some embodiments of the invention, the cell is a genetically modified cell.

According to some embodiments of the invention, the spherical particles are of an average size of 30-1000 nm.

According to some embodiments of the invention, the spherical particles further comprise synthetic lipids.

According to some embodiments of the invention, the spherical particles are attached to a synthetic polymer at an external surface thereof.

According to some embodiments of the invention, the spherical particles are devoid of the cytoplasmic content of the cell.

According to some embodiments of the invention, the spherical particles are liposomes.

According to some embodiments of the invention, the cell is a mesenchymal stem cell.

According to some embodiments of the invention, the topoisomerase 1 inhibitor is topotecan, irinotecan (or SN-38) and/or belotecan.

According to some embodiments of the invention, the PARPi is selected from the group consisting of Talazoparib, Niraparib, Rucaparib, Olaparib and Veliparib.

According to some embodiments of the invention, the composition further comprises, as a pharmaceutical agent, and chemotherapy and/or an immune-modulator.

According to some embodiments of the invention, the immune-modulator is a checkpoint modulator.

According to some embodiments of the invention, the immune activating chemokine or cytokine comprises RNA or DNA encoding IL-12.

According to some embodiments of the invention, the immune activating chemokine or cytokine comprises an IL-12 polypeptide.

According to some embodiments of the invention, the IL-12 is encapsulated by the particles.

According to some embodiments of the invention, the immunogenic agent is a viral peptide or RNA encoding same.

According to some embodiments of the invention, the pharmaceutical agent comprises Poly (ADP-ribose) polymerase (PARP) inhibitor (PARPi) and a topoisomerase 1 inhibitor in the same of different particles.

According to some embodiments of the invention, the immunogenic agent is an MHC-restricted peptide.

According to some embodiments of the invention, the cell expresses an exogenous antigen presenting molecule.

According to some embodiments of the invention, the cell is a tumor cell.

According to some embodiments of the invention, the composition is provided for use in treating cancer or generally as a medicament.

According to some embodiments of the invention, the cancer comprises an aberrant DNA repair pathway.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration showing main steps in derivation of NGs from MSCs according to some embodiments of the invention;

FIGS. 2A-B is a block diagram showing major steps in the process of large-scale NG production according to some embodiments of the invention (B) as compared to the process described in WO2011/024172 (A);

FIGS. 3A-B show the optimal population doubling level (PDL) for NGs production from mesenchymal stem cells (MSCs). (A) Change in MSCs morphology across passages and PDLs. (B) NGs protein (BCA) and lipid yields across PDLs.

FIGS. 4A-C show NGs production from MSCs expanded in 2D CellStack vs 3D Sartorius Bioreactor.

(A) Sartorius 2L Biostat B bioreactor that was used for expansion of MSCs. (B) Percent uptake of PKH67 labeled NGs (a lipophilic dye that penetrates and stains the membrane of the vesicle) into Jurkat cells. NGs were produced from MSCs grown in 2D CellStack (CS) or 3D 2-liter Biostat B Sartorius bioreactor. fPL=fluorescent particles. (C) Lipid yield from 9 independent NGs production runs that were initiated from MSCs grown in 2D CellStack (CS) or 3D Ambr250 or 2-liter Biostat B Sartorius bioreactors.

FIGS. 5A-B show the effect of hypotonic treatment time on cell viability and lipid yield post cell shearing. (A) Cell viability as determined by the NC-200 cell counter. (B) Lipid content as determined by Abcam ab234050 phospholipid assay;

FIG. 6 is an image showing microscopic imaging of MSCs following hypotonic treatment and shearing.

FIGS. 7A-B show lipid yield, Na/K ATPase enrichment and elimination of nuclei in NGs production process. (A) Lipid and Lamin (nuclei marker) assessment following MSCs breakdown by different shears (flow shear, homogenization, and sonication). (B) Na/K ATPase (cell membrane marker) to Lamin ratio and lipid yield in original process vs improved process that includes flow shear and elimination of nuclei by centrifugation prior to downsizing (see FIGS. 2A-B).

FIGS. 8A-B show optimization of flow shear cycles. CD29 and CD105 cell membrane labeling with fluorescently labeled antibodies was carried out prior to NGs production. Following each flow shear cycle and centrifugation, DAPI was added and fluorescent signal of CD29/CD105 (Ex/Em 490/527) (A) and DAPI (Ex/Em 358/493) (B) was assessed in both the pellet and supernatant by the TECAN Infinite 200 PRO-M Plex ELISA reader.

FIG. 9 shows elimination of Cell Nuclei by Depth Filtration. Arrows mark released nuclei post flow shear.

FIG. 10 show preservation of NGs cell membrane marker expression and potency, while nuclei is eliminated following depth filtration. MSCs following hypotonic treatment and flow shear were centrifuged (C) or filtered through 1.2 m (D1.2), 5 m (D5), or 8 m (D8) depth filter. Suspension was tested for cell membrane (Na/K ATPase and CD29) and nuclei (Lamin B1) marker expression by Western Blotting as well as for lipid and potency (% Uptake of PKH67 labeled particles to Jurkat cells).

FIGS. 11A-B show the use of Tangential Flow Filtration (TFF) for Scalable NGs Purification. (A) Lipid and protein assessment following ultracentrifugation (NG-U; Control) or TFF at different filter cut offs [20-22 nm (NG-T20), 33-35 nm (NG-T30), and 50 nm (NG-T50)]. (B) Potency (as measured by % uptake of PKH67 labeled NGs to A549 cancer cells) of NGs enriched at the end of the production process by ultracentrifugation or TFF 50 nm.

FIGS. 12A-C show TFF diafiltration volume for maximal NGs purification. (A, B) Lipid and protein assessment following filtration of flow sheared MSCs via 8 m (F8) and 0.65 m (F0.65) filters, 50 nm TFF concentration (F0.65T) and TFF diafiltration for different diafiltration volumes (up to 22 volumes; D3-D22). (C) Potency (% uptake of PKH67 labeled NGs to Jurkat cancer cells) of F8, F0.65, F0.65T, and TFF after 10 (F0.65T-D10) or 22 (F0.65Tf) diafiltration volumes. fPL=fluorescent particles.

FIGS. 13A-C show the results of NGs obtained following downsizing by the XStream High Shear Homogenizer. Zeta View particle size (A), lipid (B) and potency (C) (% Uptake of PKH67 labeled particles to Jurkat cancer cells) assessments of the flow sheared MSC suspension that was filtered through 8 m and 0.65 m depth filters and cleared from intracellular proteins by ultracentrifugation (F0.65U) prior (0 bar; F0.65U-XS-0.2) and post (100-1,500 bar) downsizing by the XStream lab homogenizer and 0.2 m filtration. fPL=fluorescent particles.

FIG. 14 shows the stability of NGs in different formulations. NGs were formulated in different formulations (as described in Table 1) and maintained at 2-8° C. for 4 weeks or at −80° C. for up to 6 months at a concentration of 0.1 (not shown) or 1 mg lipid/mL. On each indicated time point, NGs were assessed for potency (% Uptake of PKH67 labeled NGs to Jurkat cells). fPL=fluorescent particles.

FIGS. 15A-B show stability of NGs following dry freeze. NGs (500 μL at a concentration of 100 μg/mL) underwent dry freeze lyophilization using Labconco FreezeZone 1 Dry Freezer. NGs were tested for potency (% Uptake of PKH67 labeled NGs to Jurkat cells; fPL=fluorescent particles) and Zeta View assessment of particle number, size and zeta potential.

FIGS. 16A-B show the stability of NGs loaded with captisol formulated SN-38. SN-38 solubilized in 20% Captisol was mixed with NGs and sonicated using bath sonicator (2 min with 30 min recovery at 37° C.). Loaded NGs were ultracentrifuged and resuspended in Tris Magnesium pH7.4 buffer that contains 10% Sucrose. Leakage was monitored at different time points following storage at 2-8° C., −80° C., and following dry freeze, hydration and incubation at 2-8° C. % Loaded SN-38 was measured based on SN-38 fluorescence (Ex379/Em525). (B) Potency of free SN-38 or SN-38 loaded particles assessed using the ATPlite luminescence assay on SKOV-3 cells.

FIGS. 17A-B show analyses of NGs loaded with SN-38-albumin conjugate. 2.5 mg SN-38-albumin conjugate (drug to protein molar ratio of 2:1; A) was added into 25 mL resuspension of ultracentrifuged 0.65 m filtrate (lipid concentration of ˜60 μg/mL in Tris Magnesium pH7.4 buffer) (final BSA-SN-38 concentration of 0.1 mg/mL). The mixed solution underwent downsizing by the XStream high shear homogenizer at 1,500 bar in continuous mode for 1 min. Loaded NGs were cleared from unloaded drug conjugate by 2 ultracentrifugation steps (150,000×g for 90 min and then 60 min at 4° C.). Loaded NGs (as well as free SN-38 and free SN-38 albumin conjugate) were tested for their ability to stop proliferation of SKOV-3 cells (B). SN-38 conjugate which is unable to penetrate the cells demonstrated no effect.

FIGS. 18A-B show analyses of NGs Loaded with EGFP mRNA. Cy5 labeled EGFP mRNA was added into SEC purified 0.65 m filtrate (lipid concentration of ˜60 μg/mL) at a final mRNA concentration of 0.048 mg/mL in 25 mL™ pH7.4 buffer. The mixed solution underwent downsizing by the XStream high shear homogenizer (Method 1) at 500 bar in continuous mode for 1 min. Electroporation (Method 2) under two conditions (E1: Exponential 500 Volt, or E2: Sequential Wave 300 Volt) was also tested. Loaded particles were cleared from unloaded mRNA by ultracentrifugation.

Loaded NGs (as well unloaded NGs, free EGFP mRNA, and unloaded NGs+free GFP mRNA) were tested for their ability to penetrated SKOV-3 cells in a dose dependent manner and express EGFP (A, B). (A) Dose dependent uptake of Cy5 EGFP mRNA loaded NGs into SKOV-3 cells and EGFP expression across time. (B) Representative Flow Cytometry images showing % Cy5 mRNA positive and % EGFP positive SKOV-3 cells across time.

FIG. 19 shows NGs identity characterization by flow cytometry. NGs (5 μg lipid) were stained with each of the indicated 20 nm filtered Abs (CD45 was used as negative control) using vendor recommended dilutions and incubated for 30 min, at RT protected from light. Stained NGs were purified from excess Ab using ExoSpin columns and acquired by the flow cytometer.

FIG. 20 shows spherical particles derived from MSCs express the MSC marker CD90, CD73 and CD105 as well as other surface markers.

### DESCRIPTION OF SPECIFIC EMBODIMENTS OF THE INVENTION

The present invention, in some embodiments thereof, relates to compositions comprising spherical particles and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Thus, according to an aspect there is provided a method of producing spherical particles, the method comprising:
(a) subjecting cells to a hypotonic treatment so as to obtain swollen intact cells;
(b) subjecting said swollen intact cells to flow shearing to obtain ruptured cells while avoiding nuclei lysis;
(c) filtering said ruptured cells to obtain a cell preparation devoid of nuclei;
(d) subjecting said cell preparation devoid of nuclei to size separation to obtain ghosts;
(e) downsizing said ghosts using a high shear homogenizer or microfluidizer, to obtain particles of 35-400 nm; and optionally
(f) purifying said particles to obtain said spherical particles.

As used herein “spherical particles” relates to synthetic (as opposed to naturally occurring cell derived particles, e.g., exosomes) fully closed carrier molecules comprising a spherical lipid/protein bilayer membrane derived from cell membranes, in which an entrapped liquid volume is contained. Thus, these spherical particles of the present inventionare also referred to herein as cell derived liposomes (CDLs(. The spheric particle can be dry freeze lyophilized and re-dehydrated to obtain their initial structure.

Liposomes include niosomes, transfersomes, emulsions, foams, micelles, liquid crystals, dispersions, lamellar layers and the like.

The liposomes may be unilamellar or multilamellar.

According to a specific embodiment of the invention, the liposomes are unilamellar, as determined by Cryo-TEM.

According to a specific embodiment of the invention, the spherical particles exhibit native membrane symmetry and expression of native markers.

Liposomes of the present invention are composed of a whole cell membrane fraction.

According to some embodiments, the spherical particles are referred to as NanoGhosts (NGs).

The spherical particles of some embodiments of the present invention have an expected protein to lipid ratio higher than about 0.8 w/w, e.g., 0.9-5 w/w, 0.9-4.5 w/w, 0.9-5 w/w, 0.9-4 w/w, 0.9-3.5 w/w, 0.9-3 w/w, 0.9-2.9 w/w, 0.9-2.8 w/w, 0.9-2.7 w/w, 0.9-2.6 w/w, 0.9-2.5 w/w, 0.9-2.4 w/w, 0.9-2.3 w/w, 0.9-2.2 w/w, 0.9-2.1 w/w, 0.9-2.9 w/w, 0.9-2.0 w/w, 0.9-1.9 w/w, 0.9-1.8 w/w, 0.9-1.7 w/w, 0.9-1.6 w/w, 0.9-1.5 w/w, 0.9-1.4 w/w, 0.9-1.3 w/w, 0.9-1.2 w/w, 1-2.7 w/w, 1.1-2.7 w/w, 1.2-2.7 w/w, 1.3-2.7 w/w, 1.4-2.7 w/w, 1.5-2.7 w/w, 1.6-2.7 w/w, 1.7-2.7 w/w, 1.8-2.7 w/w, 1.9-2.7 w/w, 2.0-2.7 w/w, 2.1-2.7 w/w, 2.2-2.7 w/w, 2.3-2.7 w/w, 2.4-2.7 w/w, 2.5-2.7 w/w, e.g., about 2.5 w/w (for an unloaded spherical particles).

Protein and lipid assessments can be done using methods which are well known in the art, such as BCA, Bradford and Stewart phospholipids assay.

According to a specific embodiment the protein content is determined using a BCA Protein assay or Smith assay (e.g., kits are commercially available such as from ThermoFisher) and lipid assays (e.g., kits are commercially available such as from Abcam) and are typically carried out using commercial kits according to manufacturer's instructions.

Of According to some embodiments, the protein content of hMSCs spherical particles generated according to some embodiments of the invention is at least about 2 mg/10⁹ cells (as determined by a micro BCA protein assay) to about 150 mg/10⁹ cells. The lipid content is 1-30 mg per 10⁹ cells.

As used herein the phrase “cell membrane” or “cellular membrane” (which may be interchangeably used) refers to a biological membrane, essentially that which surrounds the cell.

The use of plasma membrane is of a specific advantage since it presents proteins, which are associated with cell-to-cell interactions as well as other recognition molecules, such as receptors that bind soluble ligands and may elicit beneficial therapeutic properties such as immune modulation.

The cell membrane comprises both lipids and membrane-anchored proteins, and may be referred to herein as “whole cell membrane” to distinct from lipids alone or membrane proteins alone.

Examples of membrane proteins include, but are not limited to, integral proteins, transmembrane proteins, lipid anchored proteins and glycoproteins.

According to an embodiment of the invention, the whole cell membrane fraction also includes carbohydrates.

According to a specific embodiment the cell is a eukaryotic cell [e.g., mammalian (such as human), plant, insect cell].

According to an additional specific embodiment, the eukaryotic cell is a mammalian cell.

According to yet an additional embodiment the cell can be a primary cell (i.e., non-immortalized and at times not cultured) or a cell-line.

According to yet an additional embodiment the cell can be an embryonic cell.

According to yet an additional embodiment the cell can be a fetal, post-natal or adult cell.

According to a specific embodiment, the cell is a terminally differentiated cell.

Use of a primary cell may be advantageous for clinical use where non-cultured cells are used in autologous or non-autologous (syngeneic allogeneic or xenogeneic) settings.

According to a specific embodiment the eukaryotic cell is a stem cell.

As used herein, the phrase “stem cells” refers to cells, which are capable of remaining in at least somewhat undifferentiated state (e.g., progenitor, pluripotent or multipotent stem cells) or induced to undifferentiated (e.g., induced pluripotent stem cells (iPSCs)) for extended periods of time in culture until induced to differentiate into other cell types having a particular, specialized function (e.g., terminally differentiated cells). Preferably, the phrase “stem cells” encompasses embryonic stem cells (ESCs), induced pluripotent stem cells (iPS), adult stem cells, mesenchymal stem cells and hematopoietic stem cells.

According to a specific embodiment the stem cell is a mesenchymal stem cell.

Mesenchymal stem cells are the formative pluripotent blast cells. Mesenchymal stem cells (MSCs) give rise to one or more mesenchymal tissues (e.g., adipose, osseous, cartilaginous, elastic and fibrous connective tissues, myoblasts, cardiac like cells) as well as to tissues other than those originating in the embryonic mesoderm (e.g., neural cells) depending upon various influences from bioactive factors such as cytokines. MSCs can be isolated from embryonic yolk sac, placenta, umbilical cord, fetal and adolescent skin, blood, bone marrow, adipose and other tissues, although their abundance in the bone marrow far exceeds their abundance in other tissues. MSCs have been shown to have immunosuppressive functions in various settings, including autoimmune diseases and transplantation, rendering liposomes generated therefrom ultimate tools in inflammatory and autoimmune settings.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Mesenchymal stem cell cultures can be generated by diluting for example BM aspirates with equal volumes of Hank's balanced salt solution (HBSS; GIBCO Laboratories, Grand Island, NY, USA) or PBS that contains EDTA and layering the diluted cells over Ficoll (Ficoll-Paque; Pharmacia, Piscataway, NJ, USA). Following 30 minutes of centrifugation at 2,500×g, the mononuclear cell layer is removed from the interface and suspended in HBSS or PBS. Cells are then centrifuged at 1,500×g for 15 minutes and resuspended in xeno free Rooster Nourish-MSC-XF Expansion Medium. Resuspended cells are plated in about 25 ml of medium in a 10 cm culture dish (Corning Glass Works, Corning, NY) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) or TrypLE Select for 5 min at 37° C., replated in a 6-cm plate or T225 cell bind flasks and cultured for another 14 days. Cells are then trypsinized and counted using a cell counting device such as for example, a hemocytometer (Hausser Scientific, Horsham, PA) or NC-200 cell counter. Cultured cells are recovered by centrifugation and resuspended with 5% DMSO and 30% FCS at a concentration of with CryoStor CS5 or CS10 freezing medium tol to 2×10⁶ cells per ml. Aliquots of about 1 ml each are slowly frozen and stored in liquid nitrogen (LN2) or LN2 vapor phase.

To expand the mesenchymal stem cell fraction, frozen cells are thawed at 37° C., diluted with a complete medium and recovered by centrifugation to remove the DMSO. Cells are resuspended in a complete medium and plated at a concentration of about 2,000-5,000 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000]. An exemplary embodiment for MSCs expansion is provided in the Examples section.

MSC cultures utilized by the present invention preferably include three groups of cells, which are defined by their morphological features: small and agranular cells (referred to as RS-1, herein below), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

When MSCs are cultured using methods which are well known in the art they exhibit negative staining for the hematopoietic stem cell markers CD34, CD11B, CD43 and CD45. A small fraction of cells (less than 5%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Strol markers and are dimly positive for the CD90 marker, and the RS-2 cells are negative for all of these markers.

According to a specific embodiment the MSCs are CD73+/CD90+/CD105+ alternatively or additionally the MSCs are CD34-/CD45-/CD11b-/CD34-/CD79alpha-/CD19-/HLA-DR.

Other cells, which may be used as an effective source for whole cell membrane fraction include, but are not limited to, endothelial cells, hepatic cells, pancreatic cells, bone cells, chondrocytes, neuronal cells, immune cells (e.g., lymphocytes, e.g., T-lymphocytes), fibroblasts and the like.

The cells can be used native (i.e., not manipulated by genetic modification) or genetically modified to manipulate the membrane composition of the cell.

Such a genetic modification can ensure correct/enhance targeting. At this point, it does not refer to the loaded pharmaceutical agent as further described hereinbelow but to the ingredient from which the spherical particles are formed. Nonetheless, such as an expression product may have a therapeutic value.

The advantage of genetic modification is in its increased efficiency. Essentially all (>95%) the spherical particles generated from genetically modified cells express the gene-of-interest. The gene-of-interest may be constitutively expressed on the cell source (by integration to the cells genome) or transiently expressed (episomal expression) such as to avoid hazardous implications of stable transfection agents (e.g., lentiviral, adenoviral and retroviral vectors).

Thus, the cells may be genetically modified to express an exogenous (i.e., heterologous) gene-of-interest (i.e., either not naturally expressed in the native membrane or in order to enhance the expression of endogenous proteins that are naturally expressed on the native cell's membrane but in lower levels).

According to specific embodiments, the gene-of-interest encodes a membrane protein. The gene-of-interest may be a native membrane protein or modified to have a membrane localization signal and other motifs needed for membrane anchorage e.g., a transmembrane domain.

Examples of membrane proteins which may be heterologously expressed include, but are not limited to, a targeting protein (e.g., antibodies, receptors, membrane anchored ligands, decoys), a protein which affects the chemistry of the membrane (e.g., structural proteins, charged proteins), a diagnostic protein (e.g., an enzyme as described in length below) and a therapeutic protein (as described in length below).

A targeting moiety includes a targeting protein such as an antibody, a receptor ligand and a non-proteinecious molecule such as carbohydrates, which binds cell surface or extra-cellular matrix markers. For example, prostate-specific membrane antigen (PSMA) that is over-expressed on prostate cancer cells can be targeted by its ligand NAAG³ conjugated to a transmembranal motif (e.g, truncated LIME)⁴. This may be achieved, by genetically engineering the cells (of which the CDLs are derived from) to express the chimeric or natural form of NAAG. For example, the expression plasmid encoding LIME is constructed by PCR and subsequent insertion of the corresponding fragment into pcDNA3.1 (Invitrogen). The primers also have BamHI (5′ primer and 3′ primer) site extension to facilitate the subcloning. The PCR product is digested with BamHI and inserted into corresponding sites in pcDNA3.1(+) (CLONTECH Laboratories, Inc.). For expression vector encoding LIME-acetylaspartylglutamate (NAAG), the open reading frame can be inserted into plasmid coding LIME such that the NAAG is conjugated through its N-terminus and maintains its C-terminus free to react with PSMA [i.e., LIME(C)—(N)NAAG-COOH]. Alternatively, expression plasmid encoding NAAG-LIME chimera can be constructed following the method described previously described for CD8-LIME chimera⁵. Fragments corresponding to NAAG and LIME transmembrane region were generated by PCR. Primers encoding the 3′ sequences of the NAAG and the 5′ sequences of the LIME fragment were designed to overlap, such that annealing of the two products yielded a hybrid template. From this template, the chimera is amplified using external primers containing XbaI sites. The NAAG-LIME chimera is inserted into pcDNA3.1(+).

As used herein, the phrase “surface marker”, refers to any chemical structure, which is specifically displayed at uniquely high density, and/or displayed in a unique configuration by a cell surface or extracellular matrix of the target cell/tissue.

For example, the targeting moiety may be useful for targeting to tumor cells. For example, it is generally accepted that the intracellular environment of tumor cells is more alkaline compared to their immediate extracellular environment, which in turn is more acidic than the microenvironment found in the angiogenic blood vessels feeding the tumor. In addition, many previous studies have shown that the surface charges of tumor cells are more negative compared to benign normal cells and even less invasive tumor cells. Accordingly, it may be useful to express membrane-bound enzymes and/or proteins, which will render the liposomes with a positive charge only in the acidic intermediate extracellular environment of the tumor. For example, any membranal protein with a pI of about 7.2-7.4 that falls between the high alkaline pH of the angiogenic blood vessels (pH>7.4) and the low acidic pH of the tumor immediate extracellular environment (pH<7.2) can be used. Such proteins can be specifically identified by cross referencing the RCSB Protein Data Bank (PDB) for human plasma membrane proteins. The expected desirable pI (7.2-7.4) for those proteins can be calculated using the standard iterative algorithm that gives relatively precise results of pI calculations for raw protein sequences. The algorithm is used in the Compute pI/Mw tool at the ExPASy server. Such liposomes are expected to have negative or neutral charge in the alkaline microenvironment of the angiogenic tumor vessels and positive charge in the more acidic immediate extracellular environment of the tumor. Accordingly, this charge alteration will assist both liposomal extravasation, which is significantly enhanced for negative of neutral particles, and intra-tumor delivery which is more easily accomplished with positively charge particles.

Ample guidance regarding surface markers specifically over-expressed in diseases such as cancer, and antibodies specific for such surface markers is provided in the literature of the art (for example, refer to: A M Scott, C Renner. “Tumour Antigens Recognised by Antibodies.” In: Encyclopedia of Life Sciences, Nature Publishing Group, Macmillan, London, UK, www(dot)els(dot)net, 2001).

Diseases associated with a target cell/tissue specifically displaying a growth factor receptor/TAA surface marker which are amenable to treatment by the method of the present invention include, for example, some of the numerous diseases which specifically display growth factor receptors/TAAs, such as EGF receptor, platelet derived growth factor (PDGF) receptor, insulin like growth factor receptor, vascular endothelial growth factor (VEGF) receptor, fibroblast growth factor (FGF) receptor, transferrin receptor, and folic acid receptor.

In a preferred embodiment, the ligand is an antibody or an antibody fragment, targeting antigens specific to a receptor on a target cell. Antibodies can be monoclonal antibodies, polyclonal antibodies or antibody fragments, which are target specific. In an embodiment, the antibodies attached to the liposomes are anti-CD19, anti-CD20, or anti-CD22, for specific binding to a B-cell epitope. These antibodies or antibody fragments are typically derived from hybridomas that show positive reactivity toward the affected B-cells. It is contemplated that other antibodies or antibody fragments targeting any other cell in the body can be similarly used. For example, anti-CD19 antibodies are used to target liposome containing an entrapped agent to malignant B-cells. The antibody recognizes a unique epitope, the CD19 surface antigen, on the B-cells.

Methods of expressing heterologous proteins in eukaryotic cells are well known in the art.

Thus, an exogenous polynucleotide sequence designed and constructed to express at least a functional portion of the gene-of-interest may be expressed in the cells from which membranes are later extracted. Accordingly, the exogenous polynucleotide sequence may be a DNA or RNA sequence of the gene-of-interest.

The phrase “functional portion” as used herein refers to part of the encoded protein (i.e., a polypeptide), which exhibits functional properties of the enzyme such as binding to a substrate. For example, the functional portion of an antibody may be the variable region conferring specificity and additional/or alternatively the constant region, i.e., Fc, which may activate complement and induce cell killing. For example, cells can be transfected with genes encoding one or more members from the GPCRs family (e.g., CCR5, CXCR4 etc.) that will render the liposomes targeted against abundant of cellular pathologies including auto-immune and viral diseases (e.g., HIV/AIDS).

To express exogenous gene-of-interest in eukaryotic (e.g., mammalian) cells, a polynucleotide sequence encoding the gene-of-interest is preferably ligated into a nucleic acid construct suitable for eukaryotic cell expression. Such a nucleic acid construct includes a promoter sequence for directing transcription of the polynucleotide sequence in the cell in a constitutive or inducible manner.

Alternatively, cells, membranes, ghosts or the spherical particles derived therefrom, may be chemically treated such as to present a protein, a saccharide, a synthetic polymer, a peptide or any combination of same. Methods of modifying the membrane with a synthetic polymer are described hereinbelow. Such a chemical attachment may be effected at any stage from live cultured or suspended cells to produce the spherical particles.

For example, the spherical particles may be also chemically conjugated with folate that may further enhance their targeting and attachment to tumor cells, which are known to express higher levels of folate receptors compared to benign cells.

According to another example, it is also possible to permanently modulate the spherical particles to have a more positive surface charge by treating them with cations, salts or polycations (e.g., Polybrene®, polyethyleneimine and Poly-L-Lysine) rendering them more positive to better target the tumor angiogenic vasculature.

Non-native material can be also introduced to the surface of the spherical particles by fusion (e.g., PEG or detergent induced) with other liposomes (e.g., cell-derived or synthetic) that may be comprised of well characterized lipids, proteins and additives. Such a fusion, creating hybrid spherical particles, can be used to conjugate any moieties (e.g., targeting, therapeutic, diagnostic, stealth-rendering etc.) to the CDLs and to alter their surface properties. Synthetic polymers are typically used to prevent or reduce coagulation, increase dispersion, reduce interaction with blood components, evade non-specific uptake by the mononuclear phagocytic system and prolong the particle circulation time to a large extent thus, rendering the spherical particles with properties and features that are commonly referred to as stealth properties or long-circulating liposomes. Accordingly, the pH nano-environment at the particle surface may also be dependent upon the length of these molecules.

There are numerous polymers, which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie-polyglycolic acid' polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The most commonly used and commercially available lipids derivatized into lipopolymers are those based on phosphatidyl ethanolamine (PE), usually distearylphosphatidylethanolamine (DSPE).

A specific family of lipopolymers, which may be employed by the invention include PEG-DSPE (with different lengths of PEG chains) in which the PEG polymer is linked to the lipid via a carbamate linkage and Polyethyleneglycol distearoylglycerol. The PEG moiety headgroup preferably has a molecular weight from about 750 Da to about 20,000 Da. More preferably, the molecular weight is from about 750 Da to about 12,000 Da and most preferably between about 1,000 Da to about 5,000 Da. Two exemplary DSPE-PEG are those wherein PEG has a molecular weight of 2000 Da, and of 5000a designated herein DSPE-PEG(2000) (DSPE-PEG2k) and DSPE-PEG(5000) (DSPE-PEG5k).

Specific families of lipopolymers, which may be also employed by the invention, include C8 and C16 mPEG Ceramides (with different lengths of PEG chains) in which the PEG-Ceramides contain ester linkages between the PEG and ceramide moieties that allow the compound to be easily metabolized. The PEG moiety headgroup preferably has a molecular weight from about 750 Da to about 2,000 Da. More preferably, the molecular weight is about 2,000 Da.

Conventional post-insertion PEGylation of common liposomes requires heating or solublization in a detergent containing solution that might damage surface proteins and lead to encapsulate leakage. Therefore, spherical particles may be also PEGylated by the two following described methods or their combination. Primarily, PEGylated spherical particles will be prepared by detergent-dialysis incorporation of PEGylated lipids into the ghost cell membrane (prior to spherical particles preparation). Following, direct PEGylation of the spherical particles may be performed with monomethoxy-PEG activated by succinimidyl succinate, which has been proven to increase the transfection efficiency and reduce serum mediated inactivation of PEGylated lentiviral particles, used as gene transduction vectors¹⁶.

Chemical binding of non-proteinaceous components (e.g., synthetic polymers, carbohydrates and the like) to the spherical particles surface may be employed. Thus, a non-proteinaceous moiety, may be covalently or non-covalently linked to, embedded or adsorbed onto the spherical particles using any linking or binding method and/or any suitable chemical linker known in the art. The exact type and chemical nature of such cross-linkers and cross linking methods is preferably adapted to the type of affinity group used and the nature of the liposome. Methods for binding or adsorbing or linking the enzyme and/or targeting moiety are also well known in the art.

For example, the enzyme and/or targeting moiety may be attached to a group at the interface via, but not limited to, polar groups such as amino, SH, hydroxyl, aldehyde, formyl, carboxyl, His-tag or other polypeptides. In addition, the enzyme and/or targeting moiety may be attached via, but not limited to, active groups such as succinimidyl succinate, cyanuric chloride, tosyl activated groups, imidazole groups, CNBr, NHS, Activated CH, ECH, EAH, Epoxy, Thiopropyl, Activated Thiol, etc. Moreover, the enzyme and/or targeting moiety may be attached via, but not limited to, hydrophobic bonds (Van Der Waals) or electrostatic interactions that may or may not include cross-linking agents (e.g., bivalent anions, poly-anions, poly-cations etc.).

The cells may be cultured in order to obtain enough cells from which spherical particles can be produced in a large-scale manner.

According to a specific embodiment, the amount of cells for starting the process of producing the spherical particles is at least, 1×10⁶, 0.5×10⁷, 1×10⁷, 0.5×10⁸, 1×10⁸, 0.5×10⁹, 1×10⁹, 1×10¹⁰, 1×10¹¹, 1×10¹².

Methods of culturing cells are well known in the art.

The selection of the media and settings are at the discretion of the skilled artisan.

Typically, in order to obtain large amounts of cells, the cells are grown in suspension although two dimensional settings are also contemplated herein. The cells can be used fresh or after thawing a frozen batch.

As used herein “a suspension culture” refers to a culture in which the cells are grown free floating (not attached to any matrix) or, when adherent, attached to solid carriers such as microcarriers or beads and grown in suspension. Such carriers can be composed of macromolecules such as cellulose, dextran, agarose, or acrylamide, or inorganic materials such as glass. The surfaces of the carriers may be further modified by physical or chemical treatments, such as adsorption or covalent cross-linking of molecular entities with a desired charge or other desired characteristic. Alternatively, the carrier may consist of a cell or cells encapsulated within a matrix that allows perfusion of sub-cellular sized material. Microcarrier culture has significant advantages, including the scale-up of cultures, and also allows cell units to be conveniently exposed to selected culture conditions as required in order to cause the desired cellular process. In the broadest embodiment, therefore, the invention provides a method for culturing cells in vitro, comprising growing said cells adhered to a microcarrier or bead.

According to a specific embodiment, the cells are selected having a population doubling level (PDL) which would increase yield of spherical particles.

According to a specific embodiment, the expansion is carried out following thawing a a working cell batch and expansion under xeno-free growth conditions in a controllable bioreactor. Such conditions can be further scaled-up such as to a 50 L or larger reactors (e.g., of Biostat STR® reactors). It has already been demonstrated that MSCs can successfully be grown on microcarriers in single-use stirred bioreactors at benchtop-scale instead of the commonly used planar, one- or multiple-layer flasks, such as CellSTACKs or Cell Factories.

The cultivation process of cells, e.g., MSCs in large scale setting typically includes 3 steps: (1) initial cell attachment, (2) cell expansion, and (3) cell harvest. The microcarriers along with medium are transferred to the bioreactor (e.g., Univessel® SU) and equilibrated for at 37° C., 5% CO₂. Dynamic conditions are then applied, e.g., agitation, shaking. Afterwards, the thawed and pooled MSCs are inoculated at an initial density at the range of 10³ cells cm-². No agitation is performed for a while e.g., 2-6 h, to allow the cells to attach to the microcarriers. The reactor is then filled with medium to the maximal working volume. When present, the impeller speed is accelerated according to the discretion of the skilled artisan e.g., initially set to X e.g., 100 rpm and increased to Y e.g., 135 rpm as cultivation progresses. MSCs, for example, can be grown at 37° C., pH 7.2 and 0.1 vvm headspace aeration for a predetermined number of days, e.g, 7 days. On a predetermined day of cultivation it is possible to perform at least 50% medium exchange or by adding a fed batch to prevent nutrient limitation. Once the maximum cell density is reached (e.g., day 7 of cultivation), the cells are separated from the microcarriers such as by using a sieving procedure combined with enzymatic cell (e.g., TrypLE™ Select) detachment and washing steps. Finally, the microcarrier-free suspension is centrifuged followed by supernatant removal and cell resuspension in fresh culture medium before initiation of NGs process or alternatively vialing and freezing occurs, if so desired. An exemplary embodiment is provided in the Examples section which follows.

As used herein “population doubling level (PDL)” refers to the total number of times the cells in a given population have doubled during in vitro culture.

According to a specific embodiment, the cells (e.g., MSCs) exhibit a PDL of 15-30, e.g., 15-27, 15-25, 15-22, 15-20.

According to a specific embodiment, the cells (e.g., MSCs) exhibit a PDL of 18-20.

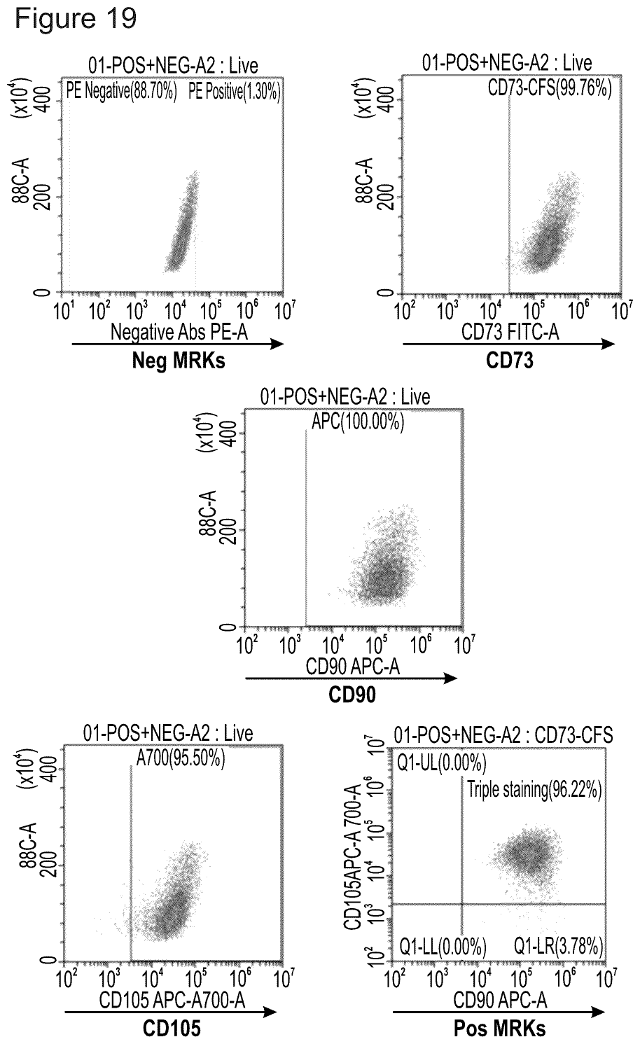

Cells, e.g., MSCs, post harvesting and prior to particle production, are characterized by flow cytometry for expression of typical markers e.g., MSCs markers and for no expression of negative hematopoietic markers (e.g., as per ISCT guidelines; see FIG. 19).

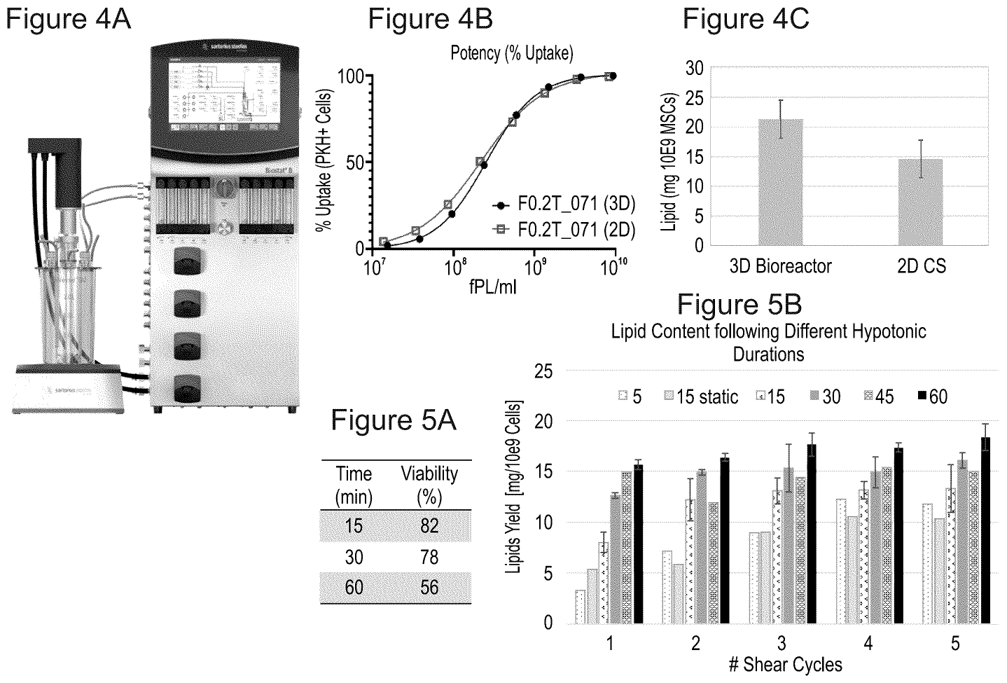

The spherical particles-derived therefrom can be evaluated by determining marker expression, e.g., MSCs surface marker expression (see above), lipid and/or protein content and/or functionally such as by particle uptake by cancer cells, see e.g., FIG. 4B. Such qualifications may help in determining the most effective culturing and production methods.

Once harvested, the cells can be used fresh or subjected to freezing procedures to be used later on (as a single batch or pooled).

According to a specific embodiment, the cells are of a single cell type, meaning that the population is pure [≥95% of the cells in culture are of a single cell type, e.g., ≥95% MSCs (as can be assessed by CD73+/CD90+/CD105+ signature)].

It will be appreciated that the present teachings do not necessitate the step of culturing the cells, but rather, the present method of producing spherical particles can be initiated with a cell preparation following culturing.

According to some embodiments, the method comprises culturing the cells prior to step (a) which is a hypotonic treatment.

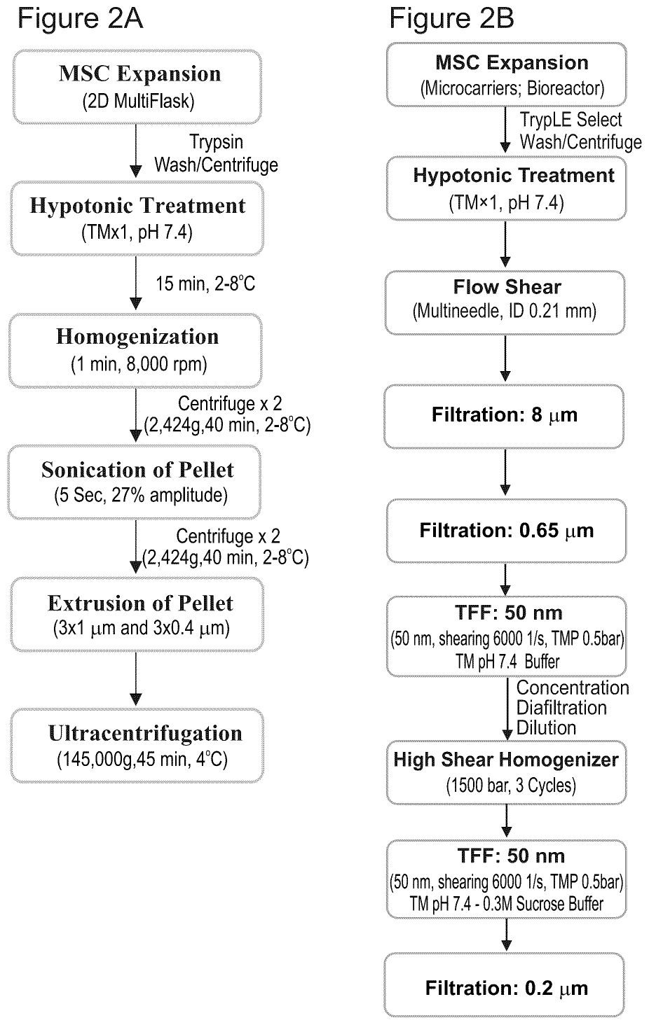

Embodiments of the method are presented in FIG. 2B.

As mentioned, a cell pellet (following centrifugation) can be subjected to hypotonic treatment to obtain swollen intact cells.

Hypotonic treatment may be the first step in the downstream processing of the cells to spherical particles. This step is intended to swell/enlarge cells and ease their shear-mediated breakdown in the following production step.

As used herein “hypotonic treatment” refers to incubating the cells in a solution that has lower osmotic pressure than that of the cells.

Measures are taken to achieve maximal cell swelling while avoiding cell membrane rupture.

According to a specific embodiment, the hypotonic treatment is done under dynamic conditions, e.g., under rotation (e.g., 10-100 rpm).

According to a specific embodiments, the treatment is effected for 10-60 minutes, e.g., 5-40 minutes e.g., 20-40 minutes, 25-35 minutes.

According to a specific embodiment, the treatment is performed under 2-8° C.

According to a specific embodiment, the osmolarity of the hypotonic treatment is at least 5 mOsm/kg, e.g., 5-100 mOsm/kg, e.g., 5-20 mOsm/kg.

According to a specific embodiment, the hypotonic solution is Tris Magnesium (TM)×1 pH 7.4. Other Examples include but are not limited to PBS, Hepes, NaPi or MgSO4, each having the indicated osmolarity.

Osmolarity can be assessed by an osmometer (e.g., 3320 Single-Sample Micro Osmometer, Advanced Instruments).

The cell's diameter and/or cell viability assays can be determined to evaluate the effect of the hypotonic treatment. For example, treatment for 15 min at 2-8° C. resulted in cell swelling to a diameter of 29.7±2.6 m (assessed by FlowCam 400, Fluid Imaging Technologies), similar to the diameter of the cells post incubation with DDW (29.7±3.0 m).

According to a specific embodiment, the hypotonic treatment results in cell swelling of at least about 20%, 30%, 40%, 50%, 60%, 70%, 80%, 90%, 1.5 folds, 2 folds, 5 folds or more, as determined by cell diameter.

Following hypotonic treatment, the cells are subjected to rupturing using flow shearing. This is in stark contrast to traditional protocols for producing cell derived liposomes as in WO2011/024172, that make use of a homogenizer and then extruder that breaks the nuclei (see also FIG. 2A). The gentle shear force of flow shearingallows release/detachment of the cell membrane without nuclei lysis. Nuclei can then be filtered out without jeopardizing lipid yield.

Thus, the swollen cells are subjected to flow shearing to obtain ruptured cells while avoiding nuclei lysis.

As used herein “flow shearing” refers to mechanical cell membrane breakage by shear forces. The advantage of this method is that it releases the intracellular content and especially the nuclei in an intact form and as such prevents the contamination of the sample with DNA.

Typically, this is done by using nanoscaled obstacles in the form of a needle, channel or tube. It is also possible to make use of a capillary pump in the lysis region to accelerate the flow and reach higher shear forces.

Avoiding nuclei lysis can be qualified by determining nuclei markers such as lamin. If no nuclei are damaged, the nucleus protein marker is not detected following flow shearing and nuclei filtration.

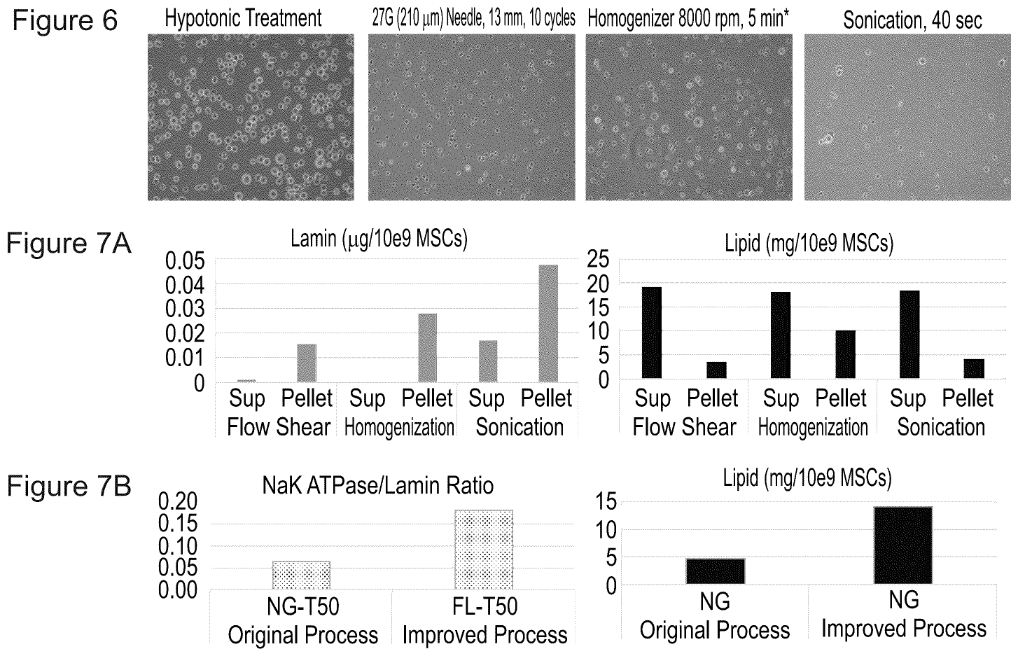

As shown in the Examples section which follows, subjecting the cells to flow shearing via a single 0.21 mm ID needle, 13 mm length demonstrated full breakdown of cells using the flow shear technique while homogenization resulted in incomplete cell breakdown and sonication resulted in nuclei dissociation (FIG. 6). The flow shear method also resulted in higher lipid yield, higher relative level of the cell membrane marker Na/K ATPase and no breakdown of the cell nuclei (FIG. 7). Hence, according to some embodiments of the method, there is no use of a homogenizer at this stage, or a sonicator.

According to a specific embodiment, the flow shearing is done with a needle/tube/channel characterized by an internal diameter (ID) of 100-400 μm and/or a needle/tube/channel length of 2-50 mm (e.g., 0.21 mm internal diameter (ID) needle, 13 mm length).

In order to improve efficacy, this step can be done using a multi-channel/needle/tube device.

The ruptured cells are filtered to obtain a preparation which is devoid of nuclei. Typically, a filter with a cut-off of, 1.2-10 μm is used, e.g., 3-10 μm, 3-8 μm, 3-9 μm, e.g., 8 μm. Such a cutoff would ensure depletion of nuclei i.e., over 90% of the nuclei.

Another step of filtration with a smaller cut-off can be employed to remove other intracellular organelles such as mitochondria, ER, Golgi and the like. In such a case a filter cut-off of 0.45-1.19 μm, 0.45-1.1 μm, 0.45-0.85 μm is used, e.g., 0.65 μm.

As opposed to the teachings of WO2011/024172 which are shown in FIG. 2A, here, the filtrate (supernatant) is collected while the pellet is discarded. In FIG. 2A, clearly the nuclei are present at this stage because the pellet is collected by centrifugation.

Following filtration, the cell preparation devoid of nuclei is subjected to size separation.

At this stage, the preparation can also be referred to as “ghosts”, as it comprises cell membranes essentially without the intracellular organelle content.

There are numerous size separation methods which can be used. These include, but are not limited to, size-exclusion chromatography (SEC), filtration, flow field-flow fractionation and deterministic lateral displacement (DLD) pillar arrays.

According to a specific embodiment, the size separation is performed by a size exclusion chromatography (SEC).

Size exclusion chromatography (SEC) is a well-established technique used for macromolecule separation based on their molecular size or hydrodynamic volumes. A typical SEC system comprises a porous stationary phase for chromatographic separation with or without coupling to a pump for elution. The commonly used stationary phase materials for membrane isolation and separation are cross-linked agarose beads (commercially named as Sepharose® (CL-2B and CL-4B) and Sephacryl® S-400). Examples of commercially available columns include, but are not limited to, IZON® qEV column, Sepharose® CL-2B column, Sepharose® CL-4B column, Sephacryl® S-500 column, Sephacryl® S-1000 column and Superdex® 200 column. Custom-made column can be used too.

According to another specific embodiment, the size separation is performed by Tangential Flow Filtration (TFF) also known as cross-flow filtration. This is an ultrafiltration (UF) technique widely used for vesicle (EV) isolation and separation. In TFF, a stream of fluids containing ghosts flow tangentially by moving across the UF membrane (hollow fiber membrane) but not directly through the membrane. Molecules smaller than molecular weight cut-off (MWCO) travel through the membrane and are discarded, while molecules larger than the cut-off level, such as ghosts, remain on the membrane and are recirculated and concentrated. TFF is more beneficial compared to conventional filters in which the fluid flows directly through the membrane, which often results in cake formation that clogs the pores. In comparison to SEC, TFF also concentrates ghosts, while SEC dilutes the isolates, making TFF suitable for large-scale ghosts isolation from diluted samples. As shown in FIGS. 11A-B, a filter cut-off of 50 nm is especially advantageous although contemplated is a range of 20 nm-100 nm or MWCO 100-750 KDa.

The TFF can be conducted using any suitable TFF device or system. For example, TFF can be conducted using a TFF system that comprises a feed reservoir, a filter device and a collection device, the feed reservoir is in fluid communication with the filter device via an inlet on the filter device, the filter device is in fluid communication with the collection device via a permeate outlet on the filter device, and the filter device is in fluid communication with the feed reservoir via a retentate outlet on the filter device.

The filter device can be in any suitable form. For example, the filter device can be in a form of a cartridge, a cassette, or a column containing a hollow fiber filter. The filter device can comprise a filtration membrane having any suitable pore size. For example, the filter device can comprise a filtration membrane having a pore size ranging from about 100 KDa to about 300 KDa, e.g., at about 100 KDa, 150 KDa, 200 KDa, 250 KDa, 300 KDa, or a subrange thereof.

The TFF can be conducted via any suitable type of process. For example, the TFF can be conducted via a diafiltration process. In some embodiments, the diafiltration process is a continuous, discontinuous, or sequential diafiltration process. TFF can be conducted via any suitable number of cycle(s). In some embodiments, the TFF is conducted via a single cycle. In some embodiments, the TFF is conducted via multiple cycles of diafiltration processes, e.g., multiple cycles of continuous diafiltration processes.

The present process or the TFF can further comprise collecting the ghosts. The ghosts can be collected using any suitable device, procedure or means. For example, the ghosts can be collected via a retentate outlet on the filter device.

The TFF can be conducted for any suitable purpose(s). See for example, Cells 2018, 7, 273; doi:10.3390/cells7120273 The TFF can also be used to concentrate and/or enrich the ghosts. In some embodiments, the TFF is used to concentrate and/or enrich the nanoparticle (s) from about 2 fold to about 1000 folds, e.g., to concentrate and/or enrich the nanoparticle (s) by 2 folds, 5 folds, 10 folds, 50 folds, 100 folds, 200 folds, 300 folds, 400 folds, or a subrange thereof.

The step of removing or reducing the level of the water-miscible solvent and the step of concentrating the ghosts can be conducted as a combined step or as separate steps. For example, steps 2) and 3) can be combined into a single step that comprises subjecting a liquid that contains the ghosts to tangential flow filtration (TFF).

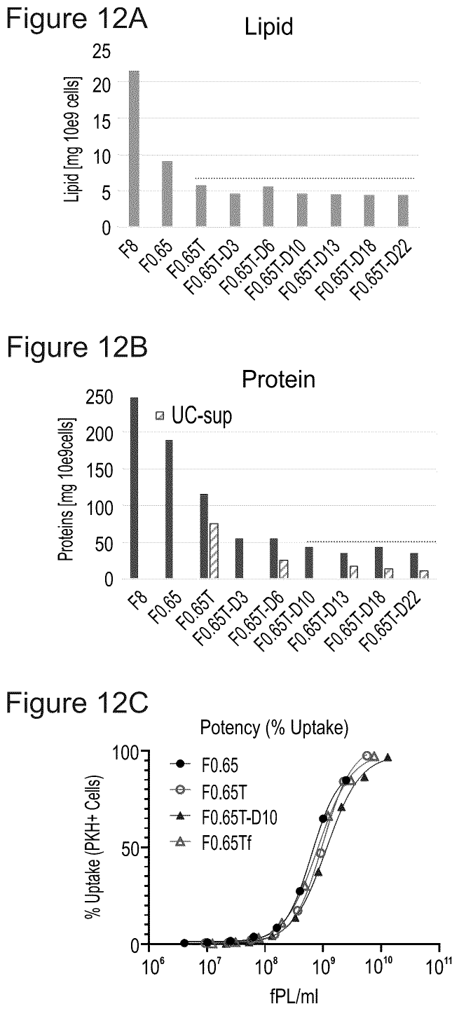

The process can be further tuned to obtain optimal TFF operating conditions (feed cross flow rate and trans membrane pressure/TMP) for maximal flux through the membrane (to minimize process time and/or membrane area and optimize pump size), such as in FIGS. 12A-B.

At this stage the ghosts are subjected to downsizing to nanometer scale. Accordingly, the method includes downsizing (or particulating, generating particles) the ghosts using a high shear homogenizer or a microfluidizer to obtain particles which are on average 35-400 nm.

According to a specific embodiment, downsizing is not performed with an extruder (by extrusion).

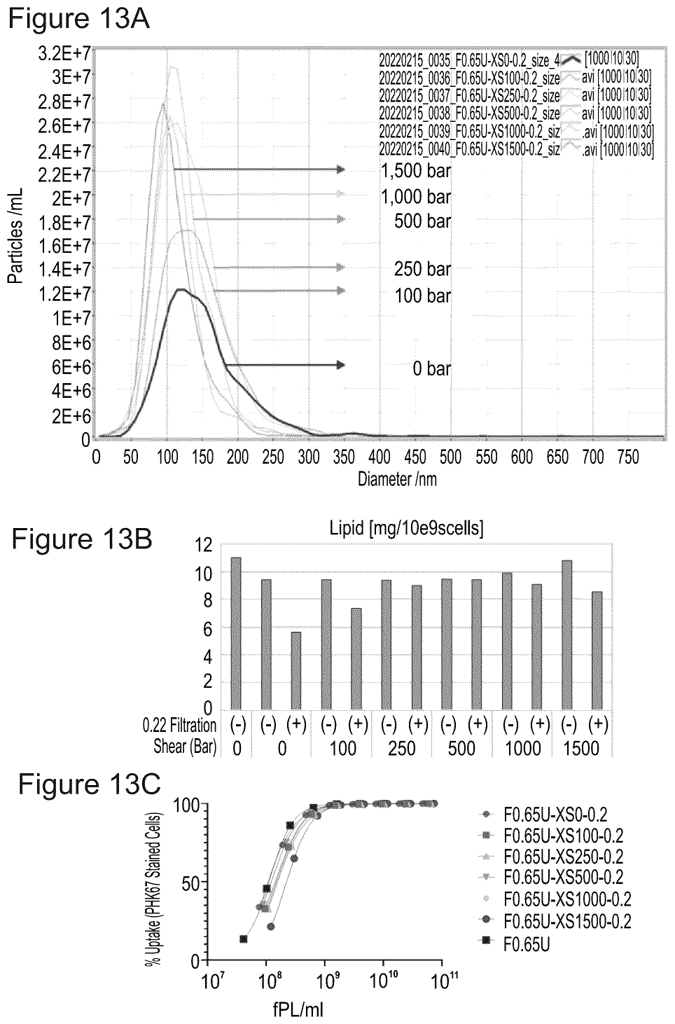

Any suitable high shear fluid processor can be used. For example, the high shear fluid processor can be a microfluidizer (or a microfluidizer processor) or a homogenizer that generates high shear force such as Benchtop GEA XStream Lab High Shear Homogenizer (with GEA NiSoX-Valve technology) which as shown in FIG. 13 was identified as a good scalable alternative to the extruder shown in FIG. 2A. As shown in FIG. 13, increased shear pressure led to decreased particle size and increase of particle uniformity. Above 250 bar, particles could pass through a 0.2 μm filter allowing sterility of the end product. Importantly, up to 1,000 bar, potency of the product was unchanged, and a marginal change was observed at 1,500 bar.

In some embodiments, the high shear fluid processor used in the present process is a microfluidizer (or a microfluidizer processor). Any suitable microfluidizer (or a microfluidizer processor) can be used. In some embodiments, the microfluidizer is configured to generate a substantially constant pressure from about 10,000 psi to about 30,000 psi, e.g., at about 10,000 psi, 15,000 psi, 20,000 psi, at about 25,000 psi, 30,000 psi, or a subrange thereof.

In some embodiments, the microfluidizer has the microfluidics reaction technology (MRT) configuration that comprises, from upstream to downstream, an inlet for inputting the nanoparticle, an intensifier pump for generating a static pressure, an impinging jet chamber for generating a high shear pressure on the nanoparticle, and an outlet for outputting the nanoparticle. In some embodiments, the microfluidizer comprises a Z-type of interaction chamber. In some embodiments, the microfluidizer comprises a Y-type of interaction chamber.

The present process can further comprise cooling the preparation. In some embodiments, the spherical particles in a channel after exiting a chamber of the microfluidizer are cooled by a product chiller that contains a coolant. The product chiller or the coolant in the product chiller can be set at any suitable temperature. For example, the product chiller or the coolant in the product chiller can be set at a temperature ranging from about 2° C. to about 8° C.

According to a specific embodiment, following high shear homogenization (e.g., at 1000-2000 bar) the preparation is subjected to another round of TFF (e.g., at 20-100 nm, e.g., 50 nm) or SEC to remove residual free protein, lipid RNA or non-encapsulating payload.

An optional step of purification can be employed such as by filtering said particles to obtain spherical particles such as with at a filter cut-off of 0.2-0.5 μm, e.g., 0.2-0.22 μm.

Once the spherical particles are formed (i.e., with or without a pharmaceutical agent, as further described hereinbelow), they may be characterized for their size distribution, composition, concentration, zeta potential, electrical surface potential, surface (local) pH, protein to lipid ratio, marker expression, and therapeutic efficacy in vitro and in vivo.

According to some embodiments of the invention, empty spherical particles or spherical particles comprising one or more pharmaceutical agent of the present invention are in the size range of 10-1000 nm, 30-500 nm, 35-400 nm, 50-200 nm, 70-150 nm.

For example, the spherical particles can have an average diameter from about 10 nm to about 1000 nm. In certain embodiments, the diameter of the nanoparticle is at least about, 20 nm, 30 nm, 40 nm, 50 nm, 60 nm, 70 nm, 80 nm, 90 nm, 100 nm, 110 nm, 120 nm, 130 nm, 140 nm, 150 nm, 200 nm, 300 nm, 400 nm, 500 nm, 600 nm, 700 nm, 800 nm, 900 nm and not exceeding 1000 nm, 900 nm, 800 nm, 700 nm, 600 nm 500 nm or any combination thereof which is considered a specific embodiment, e.g., 20-500 nm or any sub-range within about 20 nm to about 1000 nm, e.g., any range between any two of the above sizes.

An advantage of spherical particles smaller or about 100-nm is their ability to penetrate through very narrow blood vessels which is of great significance in diagnostic and treatment.

Any method known in the art can be used to determine the size of the liposome. For example, a Nicomp Submicron Particle Sizer (model 370, Nicomp, Santa Barabara, Calif.) utilizing laser light scattering can be used. Other methods of measuring liposome size include flow cytometry, photocorrelation spectroscopy, laser diffraction, low-angle laser light scattering (LALLS), medium-angle laser light scattering (MALLS), light obscuration methods (Coulter method, for example), rheology, or microscopy (light or electron).

Other methods and devices for determining size of the spherical particles include, but are not limited to, ExoView™ R100 NanoSight from NanoView (size 50-200 nM), Flow nanoAnalyzer from NanoFCM (size, concentration, phenotyping), or qNano Gold from iZon Tunable Resistive Pulse Sensing (TRPS; Size, Concentration, Charge), or ZetaView ParticleMetrix (NTA, fNTA; concentration, size, charge, phenotype).

The preferred average effective particle size depends on factors such as the intended route of administration, formulation, solubility, toxicity and bioavailability of the compound.

Values of Zeta potential in experimentally tested spherical particles are provided infra.

According to some embodiments of the invention, the average zeta potential of the spherical particles (without PEGylation or pharmaceutical agent) is (−5) to (−) 50 mV, e.g., about −30 mV.

The particles can be further qualified using methods which are well known in the art. For example, as shown in the Examples section, spherical particles derived from MSCs are characterized by flow cytometry for expression of MSC identity markers as well as markers that are found expressed on their surface using Ab array.

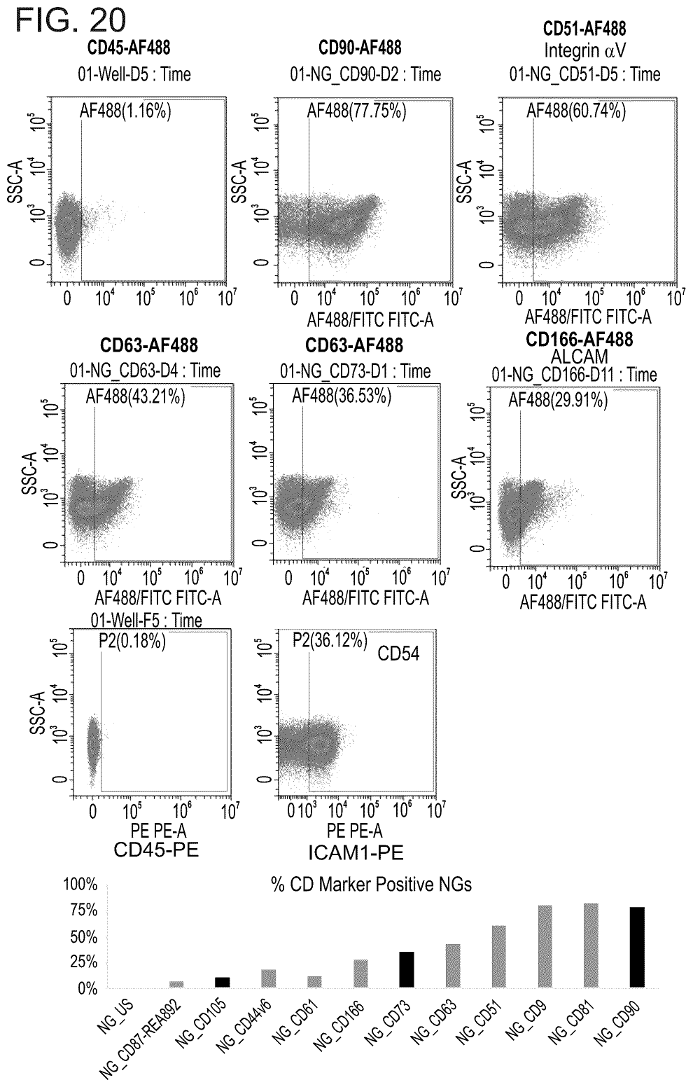

As shown in FIG. 20, spherical particles derived from MSCs express the MSC marker CD90, CD73 and CD105 as well as other surface markers.

According to an aspect of the invention, a composition comprising a plurality of spherical particles composed of a whole cell membrane fraction, wherein the spherical particles exhibit native membrane symmetry and expression of native markers obtainable according to the method as described herein.

According to a specific embodiment, the composition is characterized by a membrane to nucleus marker ratio higher by at least 1.5 fold, 1.6 fold, 1.7 fold, 1.8 fold, 1.9 fold, 2.0 fold, 2.2 fold, 2.5 fold, 2.7 fold, 3.0 fold, 3.5 fold, 4.0 fold, 5.0 fold, 7.0 fold, 10 fold, 20 fold (e.g., 2-5 fold, see for example FIG. 7B) than that obtained when using a homogenizer instead of flow shearing to rupture cells, or when collecting the centrifugal pellet following homogenization instead of the filtrate following the flow shearing.

The compositions thus obtained, are of high quality in terms of purity (e.g., level of DNA in the preparation) and as such can be beneficially used in the clinic.

These can be further formulated-frozen or used fresh.

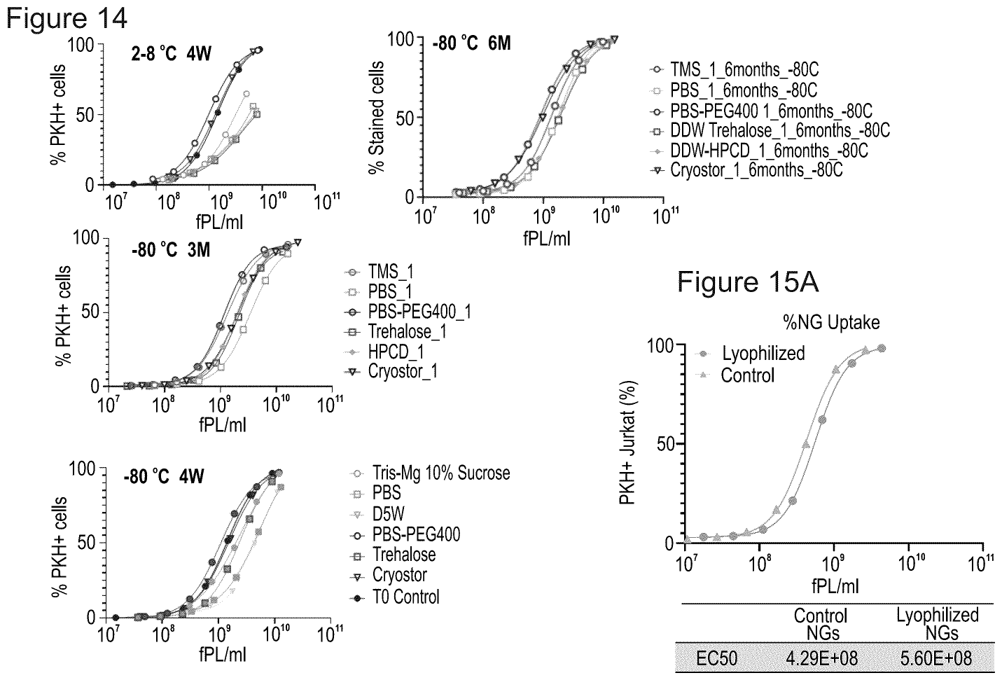

The present inventors have used different cryoprotectants/formulations that are FDA approved for IV injection. As shown in FIG. 14 and Table 1, 20% PEG400 (in PBS) and 10% Sucrose in TM pH7.4 buffer supported long term stability (for at least 6 months) at −80° C. 20% PEG400 also supported 4 weeks stability at 2-8° C. when the particles were formulated at a concentration of 1 mg Lipid/mL (FIG. 14) and 0.1 mg Lipid/mL (not shown). Further description of general formulations is provided hereinbelow.

According to a further aspect of the invention, the composition has a therapeutic property per se (especially when produced from MSCs which are known for their immunomodulatory properties), but addition of a pharmaceutical agent increases its efficacy and alternatively or additionally shifts its therapeutic value per the desired activity.

Thus, according to an aspect of the invention there is provided a method of producing a pharmaceutical composition, the method comprising producing the composition which comprise the spherical particles as described herein (and exemplified by FIG. 2B) and adding a pharmaceutical agent of interest following said size separation and prior to said downsizing so as to obtain spherical particles encapsulating said pharmaceutical agent.

Any non-encapsulated pharmaceutical agent, is removed by size separation (e.g., TFF, SEC, as described above).

As used herein the phrase “pharmaceutical agent” refers to a therapeutic agent or diagnostic agent, which can be used to treat or diagnose a medical condition, respectively.

According to a specific embodiment, the composition comprising the pharmaceutical agent and the spherical particles is hypo or non-immunogenic especially when the cell source is a mesenchymal stem cell.

Thus, the spherical particles of the present invention may have a pharmaceutical agent encapsulated therein either within the intra-liposomal polar phase or the lamellar non-polar lipid phase.

Although the present inventors have discovered that an especially beneficial method of producing the pharmaceutical composition is adding the agent to the preparation prior to the downsizing (and following the size separation) the present teachings further comprise other methods as described below.

Methods of conjugating molecules (e.g., targeting moieties, pharmaceutical agents, synthetic polymers and the like) to liposomes are well known in the art. For example, a the pharmaceutical agent (or any other molecule) may be attached, conjugated or adsorbed to surface of the liposomes, ghosts or the cells of which the liposomes derive from based on hydrophobic interactions (Van Der Waals bonds) or electrostatic interactions with or without the use of cross-linking agents (e.g. anions and poly-anions). Hydrophobic and/or amphipathic pharmaceutical agent (or any other hydrophobic and/or amphipathic molecule) may be soulibilized, partially soulibilized or partitioned into the cells, ghosts or liposomal lipid membranes with or without the use of detergent and/or by detergent dialysis. A pharmaceutical agent (or any other molecule) may be attached, conjugated or adsorbed to surface of the liposomes, ghosts or the cells of which the liposomes derive from based on covalent bonds with active groups. A pharmaceutical agent may be attached, conjugated or adsorbed to surface of the liposomes, ghosts or the cells of which the liposomes derive from as a conjugate of an antibody or part of that specifically recognized a natural moiety found on the liposomes, ghosts or cells. For example, pharmaceutical agent may be adsorbed to the surface (inner or outer) of the liposomes via, but not limited to, polar groups such as amino, SH, hydroxyl, aldehyde, formyl, carboxyl, His-tag or other polypeptides. In addition, the pharmaceutical agents may be adsorbed via, but not limited to, active groups such as succinimidyl succinate, cyanuric chloride, tosyl activated groups, imidazole groups, CNBr, NHS, Activated CH, ECH, EAH, Epoxy, Thiopropyl, Activated Thiol, etc.

Entrapped in, adsorbed, expressed, conjugated, attached, and/or solubilzed on the liposomes' surface or membrane is a therapeutic agent for delivery to the target cells and/or tissues by one or more of, but not limited to, the following mechanisms:

Direct intracellular delivery of the agent by means of membrane fusion between the liposomes and cells and/or liposomal uptake by endocytosis, receptor mediated uptake, phagocytosis or by any kind of transmembranal transport mechanism.

Diffusion and/or leakage of the agent from the liposome and consequent binding to the surface of the target cells/tissue and/or uptake into the target cell/tissue by diffusion, endocytosis, phagocytosis or by any kind of transmembranal transport mechanism.

Binding to the surface of the target cells and/or tissues of an agent which is permanently, constantly or transiently expressed, attached, adsorbed, conjugated and/or solubilzed on the liposomes' surface or membrane.

A variety of therapeutic agents can be entrapped in lipid vesicles, including water-soluble agents that can be stably encapsulated in the aqueous compartment of the liposome, lipophilic compounds that stably partition in the lipid phase of the vesicles, or agents that can be stably or transiently attached, conjugated, adsorbed or expressed on to the outer or inner surfaces of the liposomes, e.g., by electrostatic, covalent or hydrophobic interactions.

Exemplary water-soluble compounds include small molecules (i.e., less than 1000 Daltons) or large molecules (i.e., above 1000 Daltons); biomolecules (e.g. proteinaceous molecules, including, but not limited to, peptide, polypeptide, post-translationally modified protein, antibodies etc.) or a nucleic acid molecule (e.g. double-stranded DNA, single-stranded DNA, ds/ss RNA (e.g., siRNA, antisense, ribozymes), or triple helix nucleic acid molecules or chemicals. Therapeutic agents may be natural products derived from any known organism (including, but not limited to, animals, plants, bacteria, fungi, protista, or viruses) or from a library of synthetic molecules. Therapeutic agents can be monomeric as well as polymeric compounds.

As mentioned above, the therapeutic agent may be a small molecule, a protein, such as an enzyme which compensates for loss in activity or poor expression of an endogenous enzyme e.g., the enzyme hexosaminidase A, a shortage of which results in Tay-Sachs disease.

Examples of therapeutic agents but are not limited to antibiotic agents, anti-neoplastic agents, anti-inflammatory agents, antiparasitic agents, antifungal agents, antimycobacterial agents, antiviral agents, anticoagulant agents, radiotherapeutic agents, chemotherapeutic agents, cytotoxic agents, cytostatic agents, vasodilating agents, anti-oxidants, analeptic agents, anti-convulsant agents, antihistamine agents, neurotrophic agents, psychotherapeutic agents, anxiolytic sedative agents, stimulant agents, sedative agents, analgesic agents, anesthetic agents, birth control agents, neurotransmitter agents, neurotransmitter analog agents, scavenging agents and fertility-enhancing agents.

The entrapped compound may also be a diagnostic agent such as an imaging or a contrast agent as indium and technetium, enzymes such as horseradish peroxidase and alkaline phosphatase, MRI contrast media containing gadolinium, X-ray contrast media containing iodine, ultrasonography contrast media such as CO₂, europium derivatives, fluorescent substances such as carboxyfluorescein and illuminants such as N-methylacrydium derivatives.

According to a specific embodiment, the pharmaceutical agent is a water-insoluble drug.

Examples of such water-insoluble drugs include, but are not limited to: Auristatin derivatives (e.g. MMAE: monomethyl auristatin E), Maytansinoids derivatives (e.g. M1/Emtansine/Mertansine), Camptothecin analogs (e.g. deruxtecan, topotecan, irinotecan/SN-38, and belotecan), Anthracyclines (e.g. doxorubicin, daunorubicin, epirubicin and idarubicin), Platins (e.g. Cisplatin, Oxaliplatin, Carboplatin, Nedaplatin), Taxanes (Paclitaxel/Taxol/Docetaxel/Taxotere), Gemcitabine, Calicheamicin, Camptothecin and water-insoluble PARP inhibitors (e.g. Lynparza/Olaparib, Rubraca/Rucaparib, Zejula/Niraparib).

According to a specific embodiment, the water-insoluble drug is SN-38, a topoisomerase 1 inhibitor that leads to generation of DNA breaks and induction of cell death in cells undergoing proliferation. It is a highly potent drug with high systemic toxicity and compromised bioavailability. SN-38 is FDA approved as the prodrug Irinotecan (CPT-11) for colorectal cancer, as liposomal Irinotecan formulation (Onivyde, Ipsen) for pancreatic cancer and as the antibody drug conjugate (ADC) Sacituzumab Govitecan-hziy (aTrop-2/Jmmunomedics IMMU-132) for triple negative breast, bladder and urinary tract cancers. SN-38 clinical application as a small molecule drug product was hindered by poor solubility, low plasmatic stability, and severe toxicity. SN-38 ADCs overcome some of these limitations but are limited to the tumors expressing their target antigen. In addition, it is estimated that due to biodistribution, uptake and loss of conjugation in circulation, only 1-2% of ADC payload will reach the intracellular target. Embodiments of the invention that rely on various targeting moieties, allow targeted delivery to a wide array of tumors, including hard to target/penetrate brain and pancreatic tumors. It is expected that this will broaden the use of SN-38, and possibly allow delivery of higher SN-38 amounts to cancer cells, as compared to ADCs.

The following describe embodiments that aim at increasing the solubility of water insoluble drugs, while avoiding leakage from loaded spherical particles. These strategies include:

Inclusion complexation—Cyclodextrins (CD) are the versatile excipients studied extensively for pharmaceutical applications. These are cyclic oligosaccharides consisting of glucopyranose units that are united via 1,4-linkage. Three major types of CDs include α, β and γ, varying with 6, 7 and 8 glucopyranose units, respectively. CDs have a truncated-cone structure with a hydrophobic interior and a hydrophilic exterior due to the cyclic orientation of pyranose units. Central cavity of cyclodextrin is hydrophobic due to skeletal carbon atoms and ethereal oxygen. Polarity of cavity is estimated to be somewhere close to aqueous ethanolic solution. The hydrophobic nature of cavity enables entrapment of hydrophobic molecules of suitable size inside the cavity and hydrophilic surface of CD makes complex soluble in water. Apart from solubilization, cyclodextrins are also used for drug stabilization, drug protection from light, thermal and oxidative stress, taste masking of drugs, and reduced dermal, ocular or gastrointestinal irritation. The relative size of CD to the guest molecule, the presence of key functional groups on the guest molecule, and thermodynamic interactions between CD, guest molecule and solvent are the key factors that enable the formation of an inclusion complex. In addition to natural CDs, insoluble drugs are formulated using synthetic CDs like hydroxy propyl-β-cyclodextrins, hydroxy propyl-γ-cyclodextrins and sulfobutyl cyclodextrin (Captisol®), since the latter have higher solubility and safety profiles when compared to the former.

Protein/peptide-conjugation—most commonly exemplified by an antibody-drug conjugate (ADC). An ADC is comprised of a monoclonal antibody attached to a cytotoxic/water insoluble drug via a chemical linker. Examples of ADCs that have been developed and are in current clinical use include trastuzumab emtansine (T-DM1) for the treatment of metastatic HER2-positive breast cancer and HER2-positive early stage breast cancer with residual disease after neoadjuvant chemotherapy, as well as brentuximab vedotin for the treatment of recurrent Hodgkin lymphoma and anaplastic large cell lymphoma. Sacituzumab govitecan (SG), originally known as IMMU-132, is an ADC combining the humanized RS7 antibody targeting Trop-2 coupled to a proprietary hydrolyzable linker that allows for a time-dependent release of the payload, SN-38 [Drugs Today (Barc). 2019 September; 55(9): 575-585. doi:10.1358/dot.2019.55.9.3039669]. Alternatively, a highly water soluble peptide can be bound to the agent to improve its solubility and reduce its cytotoxicity, e.g., Coriat et al., International Journal of Nanomedicine 2016:11 6207-6216.

Glycosylation—Glycosylation can be used to improve solubility. Glycosyltransferase enzymes, or UGT enzymes can glycosylate almost any molecule that has the right side groups. Enzymes can work without any need to block side groups. Many of them will work stereo-specifically or with a several different sugars (glucose, galactose, xylose, glucuronic acid, rhamnose etc. Kits are commercially available such as from www(dot)gly-it(dot)com/.

Polymeric micelles—Water insoluble drugs often have greater affinity for hydrophobic solvents because of hydrophobic-hydrophobic interactions and also have affinity for hydrophobic region of micelles. Hence encapsulation of those drugs in micelles enables their formulation in aqueous vehicle. Polymeric micelles provide an advantage in terms of solubilization capacity, lower critical micellear concentration (CMC) and greater tolerability. Polymeric micelles are formed using diblcok polymers such as PEG-PLA or triblock polymers PLA-PEG-PLA. The PEG is usually the hydrophilic component in the polymer for micelles and hydrophobic chain can be of poly lactic acid, poly aspartic acid, polycaprolactic acid, etc. Due to low CMC, the polymeric micelles remain stable at low polymer concentration after dilution with body fluids. The nano-sized nature of polymeric micelles provides opportunity for tumor-targeting via enhanced permeation and retention effect (EPR). The hydrophilic PEG surface makes micelles less susceptible for reticulo-endothelial scavenging, and thus drugs have longer circulation time. Polymeric micelles can also be tailored for pH-responsive release of drugs at specific tissues and for active-targeting using targeting ligands.

Amorphous forms, solid dispersions and cocrystals—Stable crystal forms of drugs pose problem in solubilization due to high lattice energy. Thus, disordered amorphous forms offer distinct advantage over crystal forms with regards to solubility. Hence, changing the solid state characteristics of active pharmaceutical ingredient (API) renders the molecule more water soluble. Solid dispersion technology was extensively explored in recent decades for the delivery of insoluble drugs. Physically, solid dispersions are eutectic mixtures or solid solutions in which drugs exist either in an amorphous form dispersed in the carrier or as a molecular dispersion in the carrier. Solid dispersions favor enhanced dissolution of drugs due to the formation of a high-energy amorphous form or increased solubility leading to supersaturation. The increased solubility can be attributed to the dispersion of drugs at the molecular level and/or solubilization effects of the polymer. The drug remains in a metastable form for considerable time in the supersaturated state and polymeric carrier in turn can stabilize the metastable state by preventing nucleation⁵. Advances in melt-extrusion and spray-drying have accelerated industrial applications of solid dispersions for the delivery of insoluble drugs. For example, Sporanox® is a classic example of a drug (itraconazole) formulated using solid dispersion technology. At neutral pH itraconazole has a negligible solubility of 1 ng/mL. For preparing solid dispersions of itraconazole, spray-layering technology was used in which an organic solution of drug and hydroxylpropyl methylcellulose (HPMC) was sprayed over sugar beads to form a thin film consisting of molecularly dispersed drug and polymer. This amorphous formulation significantly enhanced bioavailability compared to crystalline itraconazole. Apart from spray layering, itraconazole solid dispersions were also prepared using hot-melt extrusion with varying polymers such as HPMC, Eudragit and polyvinyl pyrrolidone (PVP) mixture. In vitro studies revealed a faster dissolution of solid dispersions containing Eudragit in comparison to HPMC and sporanox. In contrast, clinical studies revealed a similarity between solid dispersions containing HPMC and sporanox, which can be attributed to the solubilization and stabilization effects of HPMC in physiological conditions (in vivo).

pH modification and salt forms—Nearly 70% of drugs are reported to be ionizable, of which a majority are weakly basic. A pH-dependent solubility is exhibited by ionizable drugs, wherein weakly acidic drugs are more soluble at pH>pKa (ionization constant) and weakly basic drugs are soluble at pH<pKa. This pH dependent solubility is used to formulate insoluble drugs. On the other hand, salt formation of weakly acidic or basic drugs provided alternate strategies for formulation of drugs which have pH dependent solubility. Pharmaceutically acceptable counter ions in the salt can provide favorable pH conditions upon dissolution in water, and thus the pH of resulting solution would be close to maximum pH of drugs. Hence salt forms may sometimes avoid pH adjustments necessary for solubilization of drugs. In addition, salt formation has been reported to improve crystallinity, stability and pharmaceutical processibility of drugs. There are many insoluble drugs on the market which are formulated with pH modification technology. Ciprofloxacin is a classic drug which is weakly basic and practically insoluble in water at neutral pH. However it exhibits pH-dependent solubility with higher solubility at acidic condition. Most of the intravenous formulations contain lactic acid as pH modifier to improve solubility.

Co-solvency and surfactant solubilization—Formulation of insoluble drugs using co-solvents is also one of the oldest and widely used technique, especially for liquid formulation intended for oral and intravenous administration. Reduction of the dielectric constant is possible by the addition of co-solvents, which facilitates increased solubilization of non-polar drug molecules. In order to maximize the solubility and prevent precipitation upon dilution, co-solvents are used in conjunction with surfactants and pH modifiers. Taxol, an intravenous injection of paclitaxel, is the most debated formulation using this approach.

Kalepu and Nekkanti 2015 Acta Pharmaceutica Sinica B Volume 5, Issue 5, September 2015, Pages 442-453 provide a detailed description of formulation strategies and is hereby incorporated by reference in its entirety.

According to some embodiments, the pharmaceutical agent is SN-38 which can be either, 1) complexed with Captisol or other cyclodextrins, or dextran (a complex branched glucan) 2) conjugated to a peptide/protein, 3) glycosylated, 4) encapsulated within micelles, e.g., polymeric micelles or organic/inorganic nanoparticles such as lipid nanoparticles—i.e. LNPs, or silica NPs, 5) bound to a linker with a functional group that can react with amine/cysteine residues on proteins, or 6) formulated as nano crystals.

Examples of RNA pharmaceutical agents include coding or non-coding RNA, e.g., mRNA coding a protein or silencing agents.

According to a specific embodiment, the mRNA or protein is an immune modulator, e.g., interleukin 12 (IL-12).

Pharmaceutical agents contemplated by the present invention include polynucleotide agents and protein agents (e.g. peptides, polypeptides and proteins).

In one embodiment, the pharmaceutical agent is one that modulates the immune response. For example, in cance the agent may be one that suppresses the immune response.

Examples of the above include, but are not limited to IL-2, G-CSF, Imiquimod, CCL3, CCL26, CSCL7, TGFBRII, IL-1, IL-6, IL-7, IL-15, IL-2, IL12, IL-18, IL21, interferon alpha, interferon beta, interferon gamma, PD-1 checkpoint binding inhibitor, CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13 or CXCL15.

In one embodiment, the therapeutic agent is a chemokine.

As used herein, the term “chemokine” refers to a family of small cytokines, or signaling proteins secreted by cells. Chemokines can be either basal or inflammatory. Inflammatory chemokines are formed upon inflammatory stimuli such as IL-1, TNF-alpha, LPS or by viruses, and participate in the inflammatory response attracting immune cells to the site of inflammation. Without being limiting, inflammatory chemokines can include CXCL-8, CCL2, CCL3, CCL4, CCL5, CCL11 or CXCL10.

In one embodiment, the pharmaceutical agent induces T-cell proliferation, promotes persistence and activation of endogenous or adoptively transferred NK or T cells and/or induces production of an interleukin, an interferon, a PD-lcheckpoint binding protein, HMGB1, MyD88, a cytokine or a chemokine.

In some alternatives, the protein is a T-cell or NK-cell chemokine. In some alternatives, the chemokine is CXCL-8, CCL2, CCL3, CCL4, CCL5, CCL11 or CXCL10. In some alternatives, the chemokine comprises CCL1, CCL2, CCL3, CCR4, CCL5, CCL7, CCL8, CCL11, CCL13, CCL17, CCL22, CCL24, or CCL26. In some alternatives, the chemokine is CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13 or CXCL15. In some alternatives, the chemokines are selected from the group consisting of EGF, Eotaxin, FGF-2, FLT-3L, Fractalkine, G-CSF, GM-CSF, GRO, IL-10, IL-12(p40), IL-12(p70), IL-13, IL-13, IL-15, I118A, IL-1RA, I1-1a, IL-1b, I1-2, I1-3, I1-4, I1-5, I1-6, I1-7, IL-8, IL-9, INF-alpha2, INF gamma., IP-10, MCP-1, MCP-3, MDC, MIP-1a, MIP-1b, PDGF-AA, PDGF-BB, RANTES, TGF-.alpha., TGF-.beta., VEGF, sCD401, 6CKINE, BCA-1, CTACK, ENA78, Eotaxin-2, Eotaxin-3, 1309, IL-16, IL-20, IL-21, IL-23, IL-28a, IL-33, LIF, MCP-2, MCP-4, MIP-ld, SCF, SDF-latb, TARC, TPO, TRAIL, TSLP, CCL1ra/HCC-1, CCL19/MIP beta, CCL20/MIP alpha, CXCL11/1-TAC, CXCL6/GCP2, CXCL7/NAP2, CXCL9/MIG, IL-11, IL-29/ING-gamma, M-CSF and XCL1/Lymphotactin.

According to another embodiment, the pharmaceutical agent is an interferon.

In still another embodiment, the therapeutic agent is an agent that inhibits a Programmed cell death protein 1.

“Programmed cell death protein 1,” or PD-1 is a protein that functions as an immune checkpoint and plays a role in down regulating the immune system by preventing the activation of T cells to reduce autoimmunity and promote self-tolerance. PD-1 has an inhibitory effect of programming apoptosis in antigen specific T cells in the lymph nodes and simultaneously reducing apoptosis in regulatory T cells. PD-1 inhibitors can be used however to activate the immune system to attack tumors and can be used to treat some types of cancers. PD-1 has two ligands PD-L1 and PD-L2. Binding of PD-L1 to PD-1 allows the transmittal of an inhibitory signal which reduces the proliferation of CD8+ T cells at lymph nodes. PD-L1 can also bind PD-1 on activated T cells, B cells and myeloid cells to modulate activation or inhibition. The upregulation of PD-L1 may also allow cancers to evade the host immune system. Therefore, inhibitors to PD-L1 or PD-1 to prevent formation of a PD-L1-PD-1 complex, is important for suppression of some cancers.

PD-1 inhibitors include antibodies such as Nivolimumab (anti PD-1) and Pembrolizumab (anti PD 1). Other checkpoint inhibitors include Ipilimumab (anti CTLA-4).

According to still another embodiment, the therapeutic agent is a chimeric receptor or a T cell receptor.

“Chimeric receptor” as used herein refers to a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain. Chimeric receptor can also be referred to as artificial T cell receptors, chimeric T cell receptors, chimeric immunoreceptors, and chimeric antigen receptors (CARs). These receptors can be used to graft the specificity of a monoclonal antibody or binding fragment thereof onto a T-cell with transfer of their coding sequence facilitated by viral vectors, such as a retroviral vector or a lentiviral vector. CARs are genetically engineered T-cell receptors designed to redirect T-cells to target cells that express specific cell-surface antigens.

The therapeutic agent may be a genome editing agent which is capable of genome editing. Examples of meganucleases include zinc-finger nucleases (ZFNs), transcription activator-like effector nucleases (TALENs using engineered endonucleases and Cas9 nucleases.

According to a specific embodiment, the pharmaceutical agent is an immunogenic agent such as a viral peptide or RNA encoding same.

According to some embodiments of the invention, the pharmaceutical agent comprises Poly (ADP-ribose) polymerase (PARP) inhibitor (PARPi) and a topoisomerase 1 inhibitor in the same or different particles.

According to some embodiments of the invention, the immunogenic agent is an MHC-restricted peptide.

As mentioned, the present inventors also contemplate polynucleotide agents as therapeutic agents. Exemplary polynucleotide agents include mRNA or RNA silencing agents which specifically hybridize to genes that are upregulated in the pathological microenvironment. Examples of such agents include siRNAs, gRNAs, antisense agents and miRNAs.

As mentioned, spherical particles of the present invention are advantageously used in the clinic.

Thus, according to an aspect of the invention there is provided a method of delivering a pharmaceutical agent, the method comprising administering to a subject in need thereof the above-describe spherical particles, wherein the pharmaceutical agent is enclosed therein or adsorbed thereon, thereby delivering the pharmaceutical agent.

According to an aspect the pharmaceutical composition is for use in treating cancer.

According to an aspect of the invention there is provided a method of treating cancer in a subject in need thereof comprising administering to the subject the pharmaceutical composition as described herein thereby treating cancer.

According to an embodiment, the cells are target cells and the spherical particles contain a targeting moiety, either chemically conjugated, heterologously added, as described above, or natively presented in the membranes from which the spherical particle is comprised (e.g., as in MSCs, which migrate to tumor cells).

The cell source for the spherical particles may be autologous or non-autologous (e.g., allogeneic, xenogeneic) to the subject.

The “target cell” referred to herein is a cell or a cluster of cells (of homogenous or heterogeneous population) and/or tissue to which a substance is to be delivered by using the spherical particle. Examples thereof include cancer cells, vascular endothelial cells of angiogenic cancer tissues, cancer stem cells, interstitial cells of cancer tissues, cells affected by genetic abnormality, cells infected by a pathogen and the like. The “target molecule” may be any molecule presented the surface of the target cells or cells adjacent to the target cells. Another form of the target molecule includes molecules which are released from cells. Examples thereof includes extracellular matrix components, secretions or architectures of cancer cells or interstitial cells of cancer tissues, and specific examples thereof include tumor markers, structures between cells and the like.

Delivering can be for diagnostic reasons (e.g., the spherical particle includes a diagnostic agent) or for treating (i.e., as a drug delivery tool, delivering a therapeutic agent) such as for treating cancer.

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a particular embodiment, the cancer is a solid tumor.

The spherical particles may be administered to the subject per se, or as part of a pharmaceutical composition.

As used herein a “pharmaceutical composition” refers to a preparation of one or more of the active ingredients described herein with other chemical components such as physiologically suitable carriers and excipients. The purpose of the pharmaceutical composition is to facilitate administration of the active ingredients to the subject.

Herein the term “active ingredient” refers to the therapeutic agent (with or without the spherical particle) accountable for the biological effect. It is to be appreciated that the spherical particle per se may have immunomodulatory function such as when prepared from membranes of MSCs or other immunomodulatory cells (e.g., immune B and T lymphocytes etc.). It is also to be appreciated that the spherical particle per se may have a cytoxoic effect on the target cells as due to membrane fusion with target cells and consequent disruption to cell membrane, cytoskeleton and functions. In such a case measures are taken to include a targeting moiety such that the cytotoxic effect becomes specific.

Hereinafter, the phrases “physiologically acceptable carrier” and “pharmaceutically acceptable carrier” which may be interchangeably used refer to a carrier or a diluent that does not cause significant irritation to the subject and does not abrogate the biological activity and properties of the administered active ingredients. An adjuvant is included under these phrases.

Herein, the term “excipient” refers to an inert substance added to the pharmaceutical composition to further facilitate administration of an active ingredient of the present invention or to increase shelf-life stability. Examples, without limitation, of excipients include calcium carbonate, calcium phosphate, various sugars and salts and types of starch, cellulose derivatives, gelatin, vegetable oils, EDTA, EGTA, Poly-L-Lysine, polyethyleneimine, Polybrene (hexadimethrine bromide), polyethylene glycols and other poly or single anions. The pharmaceutical composition may advantageously take the form of foam, aerosol or a gel.

Techniques for formulation and administration of drugs may be found in “Remington's Pharmaceutical Sciences,” Mack Publishing Co., Easton, PA, latest edition, which is incorporated herein by reference.

Suitable routes of administration include any of various suitable systemic and/or local routes of administration.

Suitable routes of administration may, for example, include the inhalation, oral, buccal, rectal, transmucosal, topical, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes, Catheterization with or without angio balloons; and/or the route of direct injection into a tissue region of the subject.

The pharmaceutical composition may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration via the inhalation route, the active ingredients for use according to the present invention can be delivered in the form of an aerosol/spray presentation from a pressurized pack or a nebulizer with the use of a suitable propellant, e.g., a fluorochlorohydrocarbon such as dichlorodifluoromethane, trichlorofluoromethane, dichloro-tetrafluoroethane; carbon dioxide; or a volatile hydrocarbon such as butane, propane, isobutane, or mixtures thereof. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

A pharmaceutical composition for parenteral administration may include an aqueous solution of the active ingredients in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or spherical particles. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition may also be formulated in rectal compositions such as suppositories or retention enemas, using, e.g., conventional suppository bases such as cocoa butter or other glycerides.

The pharmaceutical composition should contain the active ingredients in an amount effective to achieve disease treatment.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture and in vivo assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma or brain levels of the active ingredients which are sufficient to achieve the desired therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of the composition to be administered will be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### EXAMPLES

Reference is now made to the following examples, which together with the above descriptions illustrate some embodiments of the invention in a non limiting fashion.

### Materials and Methods

Process Specific Equipment
NC-200 Cell Counter (Chemometec)
BIOSTAT B-DCU II Bioreactor with BioPAT DCU Tower (Sartorius)
Bottle/Tube Roller (88881-004, Thermo Scientific)
KR2i TFF system (Repligen)
Pressure Transducer (ACPM-799-01N, Repligen)
Polar Accel 250 LC, Recirculating Chillers (Thermo Scientific)
XStream Lab Homogenizer 2000 (GEA)
CytoFlex Flow Cytometer (Beckman Coulter)
ZetaView (PMX-120-12D-R4, Particle Matrix)
Infinite 200 PRO-M Plex ELISA Reader (Tecan)
T25 easy clean control homogenizer with S25N-8G dispersing tool (IKA)
Sonicator with 3 mm microtip probe (Vibra Cell VCX750)
Extruder 10 mL or 800 mL (Evonik)
Optima MAX-XP Ultracentrifuge (Beckman Coulter)
Ultracentrifuge rotor TLA-55 (Beckman Coulter)
Ultracentrifuge rotor MLA-55 (Beckman Coulter)
Bath Sonicator, Elmasonic S10 H (Elma Schmidbauer GmbH)
Fraction Collector AFC-V1 (IZON)
Electroporator (45-2040, BTX Gemini X2)
Consumables
Via-cassettes (Chemometec 941-0011)
CellBind T225 flasks (Corning 3293)
10-Layer CellStack (CS10, Corning 3320)—For 2D MSCs expansion
2 Liter Univessel SU (Sartorius SU DUO002LL-SS01-V) and Synthemax II microcarriers (Corning CLS4622)—For 3D MSCs Expansion
27 Gauge ½-inch needle (300635, BD) or 27 Gauge 5 mm 4 Channel Multi Needle
(4MN27G-5, Iwashita Engineering)
50 mL syringes (300865, BD)
Peristaltic pump tubing (#16), PharmaPure TD=3.2 mm (AL242607, Saint-Gobain)
Sartopure PP3 capsule size 4, 100 μm (5051398P4-SS, Sartorius)
Sartopure PP3 capsule size 4, 8 m (5051301P4-SS, Sartorius)
Sartopure PP3 capsule size 4, 0.65 μm (5051305P4-SS, Sartorius)
Sartopore 2 PES capsule size 4, 0.2 m (5441307H4-00-B, 0.45/0.2, Sartorius)
Hollo Fiber TFF, MicroKros 190 cm² 0.05 m (D02-S05U-05-S, Repligen)
For 10 mL Extruder: Nuclepore Membrane Circles, 25 mm 1.0 m (110610, Whatman) and Nuclepore Membrane Circles, 25 mm 0.4 m (110607, Whatman)
For 800 mL Extruder: ipPORE Membrane, 90 mm 1.0 m (1000M25/721M101/90, A.M.D Manufacturing) and Nuclepore Membrane Circles, 90 mm 0.4 m (10417118, Whatman)
qEV-70 single size exclusion column (SP2, IZON Sciences)
1 mm Electroporation cuvette (BTX-450124)
Reagents and Solutions
RoosterNourish-MSC-XF medium [RoosterBio kit KT-016 that contains 500 mL bottle of RoosterBasal-MSC (RoosterBio SU-022) and 10 mL bottle of RoosterBooster-MSC-XF (RoosterBio SU-016)]
Rooster Replenish (RoosterBio, SU-023)—For Bioreactor 3D Growth
TrypLE Select (Invitrogen 12563-029)
Tris Magnesium (TM)×1 pH 7.4 Hypotonic Buffer 100 KDa filtered
TM×1 pH 8.6 Hypotonic Buffer
TM×1 pH 7.4 0.3M Sucrose Buffer 100 KDa filtered
60% Sucrose Solution (w/v)
SN-38 (S4908, S4908-02 Selleckchem)
Sulfobutylether-β-cyclodextrin (SBE-3-CD; Captisol) (S4592, S4592-08, Selleckchem)
Assay Kits and Antibodies
PKH67 Green Fluorescent Cell Linker Kit for General Cell Membrane Labeling (MINI67, Sigma)
Phospholipid Assay Kit (ab234050, Abcam)
Micro BCA Protein Assay Kit (23235, Thermo Scientific)
Human Lamin A/C (LMNA) ELISA kit (CSB-EL013003HU, CUSABIO)
ATP1A1 ELISA Kit (Human) (OKCA00818, Aviva Systems Biology)
Anti CD9-FITC (Clone HI9a, 312103, Biolegnd)
Anti CD29 AF488 (Clone P5D2, FAB17781G, R&D Systems)
Anti CD44 AF488 (Clone 2F10, FAB3660G, R&D Systems)
Anti CD45 AF488 (Clone 2D1, FAB1430G, R&D Systems)
Anti CD45 PE (Clone 2D1, R&D Systems)
Anti-integrin αV (CD51) AF488 (Clone P2W7, FAB1219G, R&D Systems)
Anti ICAM-1 (CD54) PE (Clone REA266, 130-120-780, Miltenyi Biotec)
Anti CD63 AF488 (Clone 460305, IC5048G, R&D Systems)
Anti CD73 AF488 (Clone 606112, FAB5795G, R&D Systems)
Anti CD81-FITC (Clone JS-81, 561956, BD Pharmingen)
Anti CD87-FITC (Clone REA892, 130-114-891, Miltenyi Biotec)
Anti CD90 AF488 (Clone Thy-1A1, FAB2067G, R&D Systems)
Anti CD105 AF488 (Clone 166707, FAB10971G, R&D Systems)
Anti CD166 AF488 (Clone 105902, FAB6561G, R&D Systems)
Anti-Sodium Potassium ATPase (Clone EP1845Y, ab76020, Abeam)
Anti-Lamin B1 antibody (ab16048, Abeam)
Anti CD29 (Integrin beta 1) antibody (ab52971, Abeam)
Goat Anti-Rabbit IgG H&L HRP (ab6721, Abeam)
ATPlite Luminescence Assay System (6016943, PerkinElmer)
Cells
Human bone marrow MSCs (RoosterBio MSC-031, Lot #00227, P2, PDL 8.8 or Lot #00238, P2, PDL 9.3 or in house der)
Jurkat T Leukemia cells (TIB-152, ATCC)
PBMCs, Donor #1 (NG-PBMC-001)
SKOV-3 Ovarian Cancer (HTB-77, ATCC)
A549 Human Lung Carcinoma (CCL-185, ATCC)

### Methods

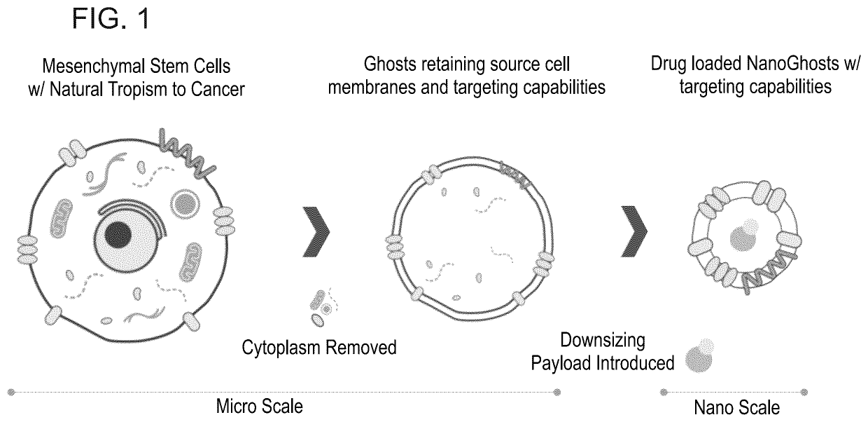

Main steps in NGs production process are described in FIG. 1. In brief, MSCs undergo hypotonic treatment followed by cell breakdown and serial filtrations steps to eliminate intracellular content while sparing cell derived vesicles at different sizes. Vesicles are then downsized to a nanomolar scale and enriched by filtration.

Each step in the originally developed NGs production process (described in FIG. 2A and below) was optimized to allow scalability and control. In addition, process steps that demonstrated high lipid loss, or alternatively, contamination with intracellular components (mainly the nuclei), were optimized/changed in order to maximize lipid yield and/or minimize intracellular contamination.

Newly developed production process (described in FIG. 2B and below) starts from ˜1E9 MSCs. For clinical Phase I study we aim at starting the process from 20-25E9 MSCs using similar larger scale systems (i.e. 50 liter STR bioreactor, continuous flow centrifuge, 10-20 multineedle device etc).

Originally developed NGs production process is briefly describe in FIG. 2A and below:

NG production was carried out with ˜50E6 MSCs/run. MSCs used in NGs production process were thawed and expanded under xeno-free growth conditions. Cells were harvested using TrypLE Select, centrifuged and resuspended in hypotonic buffer.

Hypotonic Treatment: 50e6 MSCs were suspended in 10 mL cold (2-8° C.) TM×1 pH 7.4 buffer for 15 min at 2-8° C. under static conditions.

Cell Breakdown by Homogenization: Following hypotonic treatment, MSCs underwent homogenization for 60 see, at 8,000 RPM using IKA T25 homogenizer with S25N-8G dispersing tool. Sucrose solution (60% w/v) was added to the 10 mL of homogenized cell suspension to a final concentration of 0.3 M in order to counter the hypotonic conditions. Then homogenized cell suspension was centrifuged twice (2,424 g for 40 min at 2-8° C.; Pellets 1-2) and pellet was resuspended in TM pH7.4-0.3M Sucrose buffer.

Sonication: Resuspended Pellet 2 from the previous step underwent sonication for 5 Sec, at 27% amplitude using the Vibra Cell VCX750 sonicator. Then sonicated suspension was centrifuged twice (2,424 g for 40 min at 2-8° C.; Pellets 3-4) and pellet was resuspended in TM pH 8.6-0.3M Sucrose buffer.

Downsizing by Extrusion: Resuspended Pellet 4 from a single run (or combined from parallel runs) was extruded through 1 m filter and then through 0.4 m filter using the 10 mL (or 800 mL) extruder.

Extruded solution underwent ultracentrifugation (UC) (8.9 mL/tube, 145,000 g, 45 min, 4° C.).

UC pellet was resuspended in TM pH 7.4-0.3M Sucrose buffer.

NGs were be stored at −80° C.

Newly developed NGs production process is carried as briefly describe in FIG. 2B and below:

NG production was carried out with 1×10⁹ MSCs/run. MSCs used in NGs production process were thawed and expanded under xeno-free growth conditions in Sartorius Biostat B DCU 2-liter bioreactor. Cells were harvested using TrypLE Select, washed, filtered through 100 m Sartopure PP3 capsule size 4 filter, centrifuged and resuspended in hypotonic buffer.

Hypotonic Treatment: Cells (5e6 MSCs/mL in cold (2-8° C.) TM×1 pH 7.4) underwent hypotonic treatment for 30 min at 2-8° C. under rotation (50 rpm).

Cell Breakdown by Flow Shear: Following hypotonic treatment, MSCs underwent cell breakdown by flow shear (instead of homogenization) through a single or 4 Channel Multi Needle (4MN) device (27G ID=0.21 mm, 5-15 mm long) using the TFF peristaltic pump with tubing #16 (ID 3.2 mm) at a flow rate of 120-200 mL/min. Then, instead of centrifugation and sonication, flow sheared suspension was filtered through a 8 μm Sartopure PP3 capsule size 4 filter at 100 mL/min (to get rid of cell nuclei), 0.65 m Sartopure PP3 capsule size 4 at 100 mL/min (to get rid of mitochondria and large intracellular particles), and then using Tangential Flow Filtration (TFF, MicroKros 190 cm2 0.05 μm, flow rate of 265 mL/min for shearing rate of 6000 1/s, and TMP 0.5 bar), was concentrated 10-fold and then purified by diafiltration with 10-20 TM×1 pH7.4 buffer diavolumes (10-20 times the volume of concentrated suspension, to eliminate intracellular proteins). As an alternative to TFF, size exclusion was used for purification of ghosts from intracellular proteins. Downsizing by High Shear Homogenizer: TFF concentrated and cleared suspension was diluted to original volume with TM×1 pH7.4 buffer and underwent size reduction using the GEA XStream Lab Homogenizer at 1,500 bar by passing it 3-5 independent times with overlap.

Downsized solution underwent 10-fold concentration by TFF concentration (flow rate of 265 mL/min for a shearing rate of 6000 1/s, and TMP 0.5 bar), diafiltration with TM pH 7.4-0.3M Sucrose buffer and sterile filtration via 0.2 m filter.

NGs were stored at −80° C.

Lipid and Protein Assessments:

Protein (Micro BCA Protein Assay Kit 23235, Thermo Scientific) and lipid assays (Phospholipid Assay Kit ab234050, Abcam) are carried out according to manufacturer's instructions.

Zeta View Assessment of Particle Size, Concentration and Zeta Potential:

NGs are diluted in Tris Magnesium pH 7.4 buffer to 5 E7-5E8 Particles/mL and measured for size distribution, concentration and Zeta Potential using the Zeta View.

NGs Potency Assay (using PKH67):

NGs product (˜50 μL; ˜ 5 μg lipid) is stained with PKH67 (50 μL diluted solution) as per manufacturer's instructions (PKH67 Green Fluorescent Cell Linker Kit for General Cell Membrane Labeling, MINI67, Sigma).

NGs and PKH67 solution is incubate for 30 min at 25° C. and residual dye is removed by Amicon Ultra (0.5 mL, MWCO 10 kDa, 10,000×g, RT, 10 min, 3 cycles).

Lipid assessment is carried on the Amicon Ultra filtered material.

Potency assay is carried out on Jurkat cells (20,000 cells/mL) in a 96-well plate.

Cells are incubated with labeled NGs at increased lipid or particle concentrations (range of ˜10E7-10E12) for 20 hrs, washed and acquired by the CytoFlex flow cytometer.

NGs Identity Assessment by Flow Cytometry:

NGs are assessed for expression of various CD markers by flow cytometry as follows.

Antibodies to the specific CD markers (5-20 μL, according to manufacturer's instructions) are diluted in 100 μL PBS and filtered through a 20 nm syringe filter.

NGs (5-10 μL, ˜5 μg lipid) are added to 50 μL of each filtered antibody solution and incubated at RT for 30 min in the dark.

Stained NGs are purified using ExoSpin columns and eluted with 180 μL Tris Magnesium pH 7.4 buffer or alternatively washed twice by ultracentrifugation (150,000 g, 45 min 4° C.).

Purified NGs (−1 μg/mL) are acquired using the CytoFlex flow cutometer under parameters suitable for detection of Nanoparticles.

### Example 1

### Expansion of Mesenchymal Stem Cells (MSCs) and Assessment of Population Doubling Level (PDL) for NGs Production

### Materials and Methods

RoosterBio MSCs (MSC-031) at Passage 2 (P2) and PDL 8.8 were expanded to Passage 3 (P3) on T225 CellBind flasks. When 75-80% confluency was reached at day 4, cells were harvested and seeded on a 10-layer CellBind CellSTACK multiflask and on T225 CellBind flask. Cells from passage 4 (P4) were further expanded to P5, and then P6, on T225 CellBind flasks. P6 cells were not further expanded due to reduced growth rate and change in morphology. After each harvest, the PDL was calculated using the following equation and this number was added to the PDL at seeding for calculation of cumulative PDL:

$$PDL = \frac{Log B - Log A}{Log 2}$$

Cells from each passage were cryopreserved (le6 cells/mL) for evaluation of MSC identity (CD90, CD105 and CD73) and potency (trilineage differentiation) as per International Society for Cellular Therapy (ISCT) minimal criteria (Dominicini et al., 2006 Cytotherapy Vol. 8, No. 4, 315_317).

At Passage 4 (PDL 18.4), and at Passages 5 and 6 at T225 (PDL 23 and 30, respectively), When 75-80% confluency was reached, cells were harvested, and NG production process was performed. NGs were analyzed for total protein, lipid content, and particle size, particle number and zeta potential analysis by Zeta View. Results from passages 4-6 were compared.

### Results

### MSCs Population Doubling Level (PDL) for NGs Production:

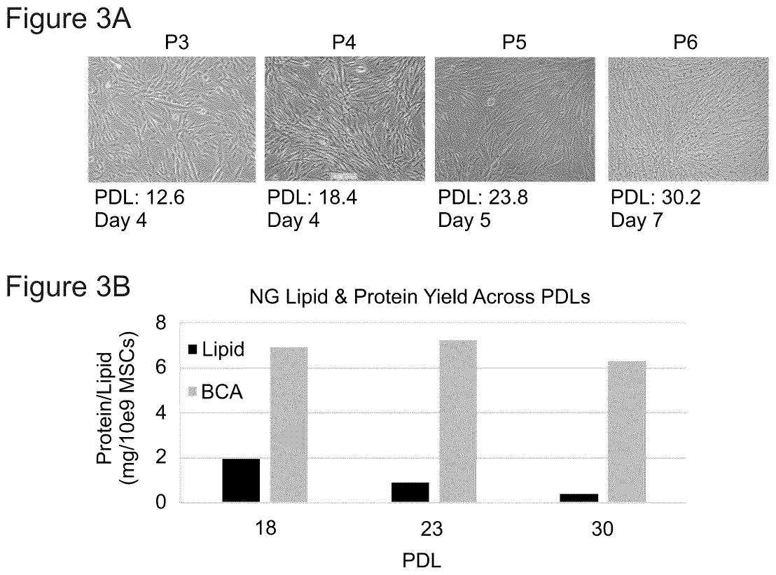

PDL (Population Doubling Level) is the total number of times cells have doubled since their primary isolation. The number of PDLs that hBM-MSCs can undergo without senescing or losing MSC characteristics, is important for determination of optimal PDL for NGs production. NGs were manufactured from MSCs that were expanded in the 2D Corning CellBind System to Passages 4 (P4), P5 and P6 with PDL ˜18, ˜23, and ˜30, respectively, and their properties were compared. The analysis of protein content and particle size revealed no change in NGs across MSC PDLs, while lipid concentration in all fractions decreased with increased PDL number (FIG. 3).

Based on these data, and the data on characterization of MSCs across PDLs showing change in morphology (FIGS. 3A-B), growth kinetics and increase in the level of negative MSC markers, albeit preserved potency, we concluded that NGs production should be initiated at Passage 4 at PDL of ˜18-20.

### Example 2

### MSCs Expansion in a Scalable Bioreactor System and NGs Production

### Materials and Methods

RoosterBio MSCs (MSC-031) at Passage 2 (P2) were expanded to Passage 3 (P3) on T225 CellBind flasks. When 75-80% confluency was reached at day 4, cells were harvested and seeded on a 10-layer CellBind CellSTACK multiflask or on the Sartorius bioreactor Ambr 250 mL or Biostat B 2L for 5-6 days. Passage 4 cells were harvested from each growth system and NGs were manufactured.

### Results

To allow scalability of the NGs manufacturing process, several bioreactor systems were tested. Sartorius bioreactor system, a well-established scalable system for expansion of MSCs, was optimized for growth of hBM-MSCs under xeno-free growth conditions using Synthemax II microcarriers. A yield of ˜0.5E6 cells/mL was reached in both the Ambr 250 mL and the Biostat B 2L Sartorius systems. NGs produced from MSCs expanded in 2D CellStack and 3D Ambr250/2L BiostatB, demonstrated preserved potency (i.e. % cell uptake to Jurkat cancer cells) (FIGS. 4A-C). Interestingly, the lipid yield of NGs produced from the bioreactor expanded cells was higher (FIGS. 4A-C).

### Example 3

### Hypotonic Treatment of MSCs

Hypotonic Buffer Tonicity: Hypotonic treatment is the first step in the downstream processing of MSCs to NGs. This step is intended to swell/enlarge cells and ease their shear-mediated breakdown in the following production step. MSCs incubation with Tris (0.01 M) Magnesium (0.001 M) (TM) buffer (osmolarity 13 mOsm/Kg as assessed by 3320 Single-Sample Micro Osmometer, Advanced Instruments) for 15 min at 2-8° C. resulted in cell swelling to a diameter of 29.7±2.6 m (assessed by FlowCam 400, Fluid Imaging Technologies), similar to the diameter of the cells post incubation with DDW (29.7±3.0 m). Based on these data, TM pH 7.4 buffer tonicity allows maximal enlargement in cell volume without affecting/compromising viability.

Hypotonic Treatment Duration: To allow the downstream processing of large cell volumes, the maximal hypotonic treatment time was assessed, as well as the effect of cell rotation. As shown in FIGS. 5A-B, viability was preserved at 30 min hypotonic treatment and decreased at 60 min. Higher lipid yield was achieved following 15 min (and 30 min) hypotonic treatment under rotating vs static condition (FIG. 5A-B). Based on these data, hypotonic treatment was extended to 30 min under rotation at 2-8° C.

### Example 4

### MSCs Breakdown

The second step in the downstream NG production process is homogenization of the cells that were treated with hypotonic buffer. Different low shearing techniques were tested and compared for their ability to release the cell membrane fraction from the intra cellular organelles. Homogenization (T25 IKA homogenizer with S25N-8G dispensing tool), sonication (Vibra Cell VCX750 with Sonics 630-0431 cup horn probe) and flow shear (via a single 0.21 mm ID needle, 13 mm length) were tested and compared. Microscopic evaluation of the cell suspension post shearing demonstrated full breakdown of cells using the flow shear technique while homogenization resulted in incomplete cell breakdown and sonication resulted in nuclei dissociation (FIG. 6). The flow shear method also resulted in higher lipid yield, higher relative level of the cell membrane marker Na/K ATPase and no breakdown of the cell nuclei (FIG. 7).

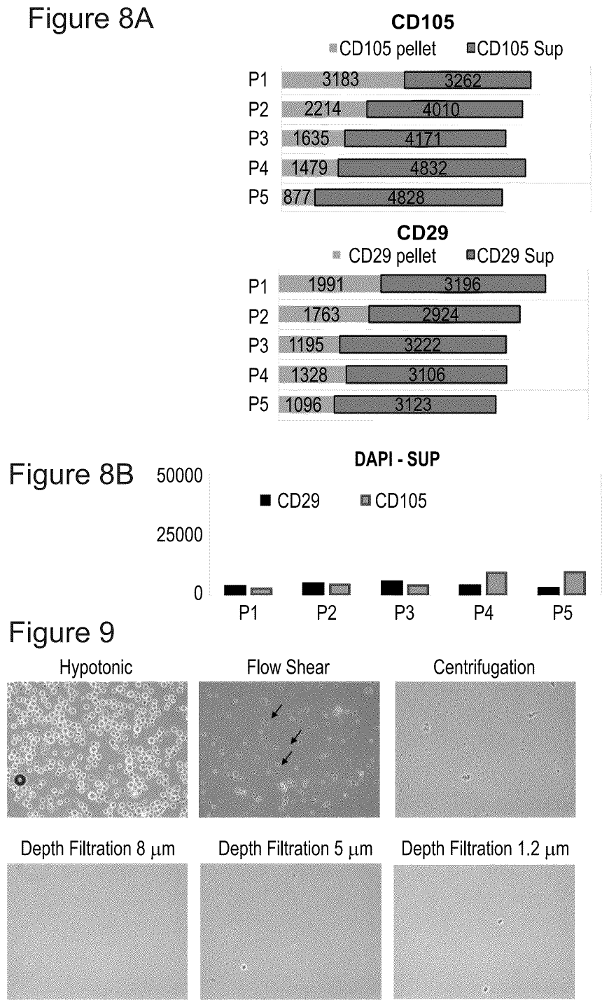

Optimization of the flow shear method was carried out using different intra needle diameters (ID) and needle lengths, at different flow shear rates and cell concentrations. These studies showed that the optimal conditions for cell breakdown are 5E6 cells/mL with a needle ID of 0.21 mm, needle length of 5-13 mm and a flow rate of 20 mL/min for a single needle and 80-150 mL/min for a 4-multineedle device (Data not shown). The number of cycles to pass via the needle for maximal release of the cell membrane while nuclei is not damaged was also assessed and based on the labeling of MSCs with fluorescently labelled anti CD29 and CD105 antibodies (CD markers of MSCs) and DAPI nuclei staining, it was concluded that within 2-3 cycles maximal yield of the cell membrane is reached while the nuclei are not dissociated (FIGS. 8A-B).

### Example 5

### Replacement of Centrifugation by Depth Filtration

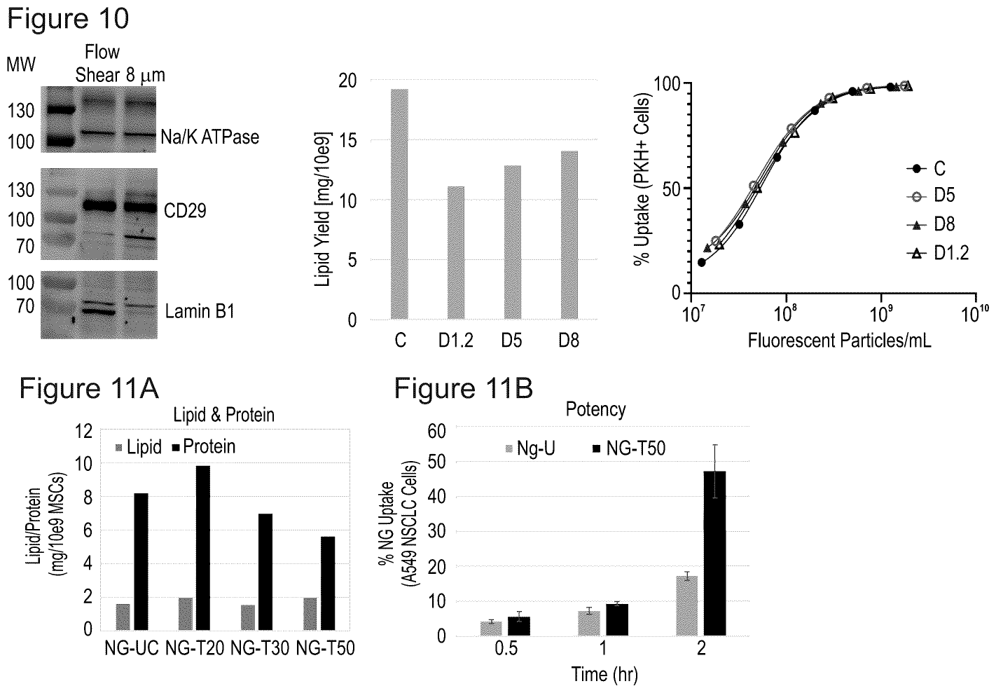

Following breakdown of MSCs by shear, nuclei and other large intracellular particles are eliminated by centrifugation (1000×g, 10 min). Depth filtration with different filter cut offs was tested as an alternative scalable method for clarification of the cell membrane fraction from the intracellular organelles. As shown in FIGS. 9-10, all filter cut offs (1.2, 5 and 8 m) cleared the nuclei, similarly to centrifugation and demonstrated preserved potency (% uptake to cancer Jurkat cells). Since 8 m clearance resulted in lower lipid loss (FIG. 10), it was selected for primary clarification of the lysed cell suspension from cell nuclei. Secondary clarification is carried out by 0.65 m depth filter, to eliminate mitochondria.

### Example 6

### Purification by Tangential Flow Filtration

Following elimination of large intracellular organelles (i.e. nuclei and mitochondria), the present inventors aimed at elimination of free intracellular proteins prior to vesicle downsizing to NGs. Tangential flow filtration (TFF) was used since it is a highly efficient scalable, and gentle method for enrichment of extracellular vesicles (EVs) from large volume samples as compared to ultracentrifugation (UC) (Bussatto et al., 2018 Tangential flow filtration for highly efficient concentration of extracellular vesicles from large volumes of fluid. Cells 12: 273).

The optimal filter cut off that allows elimination of free proteins and enrichment of lipid vesicles was determined. As shown in FIGS. 11A-B, 50 nm filter cut off allowed preserved lipid quantity while protein level was decreased to a level below ultracentrifugation. Enriched NGs demonstrated preserved potency (% Uptake to A549 non-small cell lung cancer cells) when compared to ultracentrifugation.

The present inventors then tested the optimal TFF operating conditions (feed cross flow rate and trans membrane pressure/TMP) for maximal flux through the membrane (to minimize process time and/or membrane area and optimize pump size). TMP 0.5 bar and flow rate of 27/40 mL/min were found optimal (flow rates 53 and 66 mL/min were too high for the specific pump; data not shown). Under these operating conditions, the optimal diafiltration volume for maximal reduction in intracellular protein contamination that exists following cell breakdown and filtration via the 8 and 0.65 μm filters, was tested. As shown in FIGS. 12A-C, ˜10-13 diafiltration volumes are required for clearance of free proteins. The TFF retentate post concentration and diafiltration demonstrated preserved potency (% Uptake of PHK67 labeled vesicles to Jurkat cancer cells) as compared to the original non-TFF filtrated product.

### Example 7

### Downsizing to Nanometer Scale NanoGhosts

Downsizing is carried out following clearance of flow sheared cell membrane vesicles from intracellular organelles and proteins. Previously, downsizing was carried out by extrusion via 1 μm and then 0.4 μm filters. Scaled up extrusion systems are relatively big, requiring an entire room for this step only. The present inventors have now identified Benchtop GEA XStream Lab High Shear Homogenizer (with GEA NiSoX-Valve technology) as a good scalable alternative to an extruder. As shown in FIGS. 13A-C, increased shear pressure led to decreased particle size and increase of particle uniformity. Above 250 bar, particles could pass through a 0.2 μm filter allowing sterility of the end product. Importantly, up to a shear of 1,000 bar, potency of the product was unchanged, and a marginal change was observed at 1,500 bar.

### Example 8

### Formulation of NGs

Initial studies were aimed at testing different cryoprotectants/formulations that are FDA approved for IV injection. As shown in FIG. 14 and Table 1, 20% PEG400 (in PBS) and 10% Sucrose in TM pH7.4 buffer (a cryoprotectant) supported long term stability (for at least 6 months) at −80° C. as indicated by the preserved potency/EC50 (% uptake to target Jurkat cells). 20% PEG400 also supported 4 weeks stability at 2-8° C. when NGs were formulated at a concentration of 1 mg Lipid/mL (FIG. 14) and 0.1 mg Lipid/mL (not shown).

TABLE 1
Stability of NGs in Different Formulations.

| | T0 EC50 | | | |
|---|---|---|---|---|
| | 1.3 + 09 | | | |
| | EC50 | EC50 −80° | EC50 −80° | EC50 −80° |
| Formulation | 2-8° C. | C. 4 W | C. 3 M | C. 6 M |
| 10% Sucrose in TM | 3.2E9 | 2.4E9 | 1.4E9 | 1.3E9 |
| pH 7.4 (TMS) | | | | |
| PBS (Control) | 5.8E9 | 5.3E9 | 3.3E9 | 1.7E9 |
| 20% PEG400 in | 9.8E8 | 1.1E9 | 1.2E9 | 8.9E8 |
| PBS | | | | |
| 25 mM Trehalose | 7.9E9 | 2.5E9 | 2.2E9 | 1.9E9 |
| in DDW | | | | |
| 5% Dextrose in | 6.9E9 | 4.9E9 | NA | NA |
| DDW (D5W) | | | | |
| 5% HPβCD in | NA | NA | 1.9E9 | 1.8E9 |
| DDW | | | | |
| Cryostor | 1.3E9 | 1.5E9 | 1.8E9 | 9.6E8 |

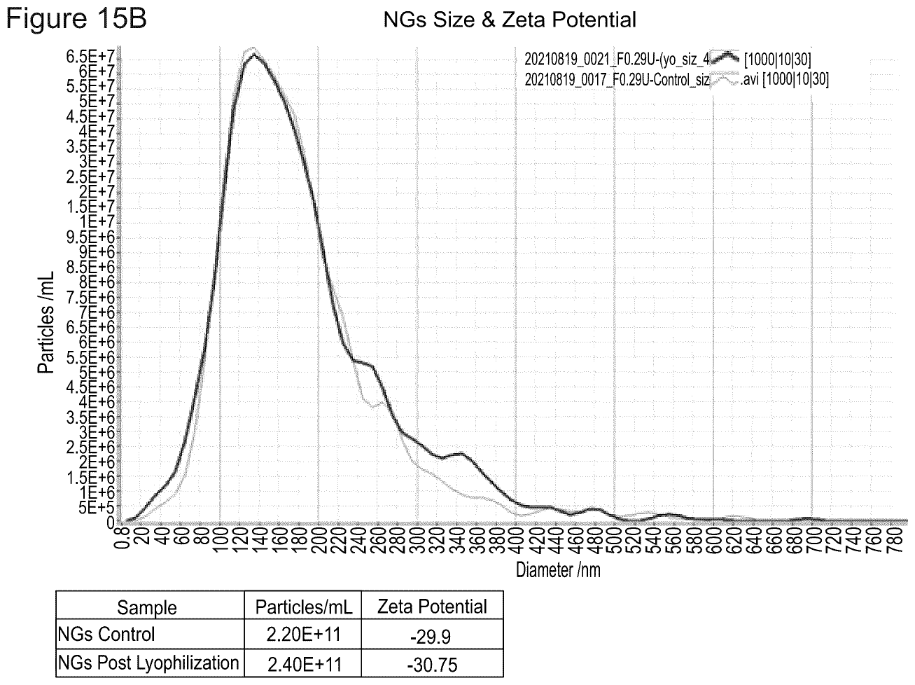

NGs were also tested for stability following dry freezing (using Labconco FreezeZone 1 Dry Freezer). As shown in FIGS. 15A-B, NGs potency, as well as NGs size, number and zeta potential were preserved following rehydration.

### Example 9

### NGs Characterization

NGs are characterized by flow cytometry for expression of MSC identity markers as well as markers that were found expressed on their surface using Ab array (not shown). As shown in FIG. 19, NGs express the MSC markers CD90, CD73 and CD105 as well as other surface markers.

### Example 10

### NGs loading with SN-38

SN-38 is a topoisomerase 1 inhibitor that leads to generation of DNA breaks and induction of cell death in cells undergoing proliferation. It is a highly potent drug with high systemic toxicity and compromised bioavailability. SN-38 is FDA approved as the prodrug Irinotecan (CPT-11) for colorectal cancer, as liposomal Irinotecan formulation (Onivyde, Ipsen) for pancreatic cancer and as the antibody drug conjugate (ADC) Sacituzumab Govitecan-hziy (aTrop-2/Immunomedics IMMU-132) for triple negative breast, bladder and urinary tract cancers. SN-38 clinical application as a small molecule drug product was hindered by poor solubility, low plasmatic stability, and severe toxicity. SN-38 ADCs overcome some of these limitations but are limited to the tumors expressing their target antigen. In addition, it is estimated that due to biodistribution, uptake and loss of conjugation in circulation, only 1-2% of ADC payload will reach the intracellular target. Embodiments of the invention that rely on various targeting moieties, allow targeted delivery to a wide array of tumors, including hard to target/penetrate brain and pancreatic tumors. It is expected that this will broaden the use of SN-38, and possibly allow delivery of higher SN-38 amounts to cancer cells, as compared to ADCs.

Since SN-38 is a highly hydrophobic drug that is not soluble in water-based solutions, several strategies are explored to increase SN-38 solubility and allow no leakage post loading to NGs. These strategies include, 1)SN-38 complexation with Captisol or other cyclodextrins, or dextran (a complex branched glucan) 2) conjugation of SN-38 to a peptide/protein, 3)SN-38 glycosylation, 4)SN-38 formulation, 5)SN-38 encapsulation within micelles, polymeric micelles or organic/inorganic nanoparticles (like: Lipid NPs—i.e. LNPs, or silica NPs), all with the optional decoration of PEG moiety, 6) binding SN-38 to a linker with a functional group that can react with amine/cysteine residues on proteins, and 7) formation of SN-38 nano crystals (directly loaded into the formed NGs or grown inside the NGs by remote loading). By controlling and manipulating different physico-chemical parameters during the encapsulation process (e.g. pH, ionic strength, temperature, drug concentrations, pressure, etc., loading can be further improved.

### Results

Cyclodextrins were reported as good FDA approved solubilization agents.

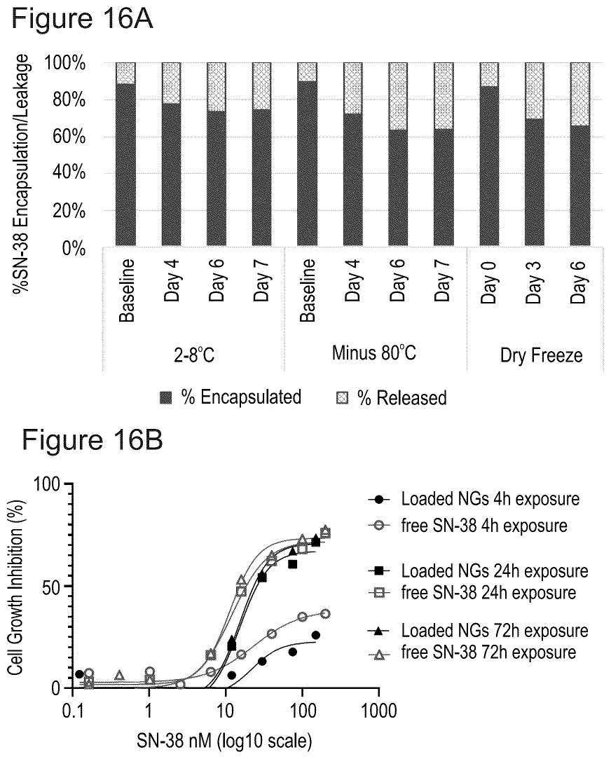

As shown in FIGS. 16A-B, SN-38 complexation/solubilization with Captisol allowed successful loading into NGs via sonication (drug/lipid ratio of 1:9). In addition, SN-38 loaded NGs were relatively stable post freeze dry and rehydration (FIG. 16A-B) and demonstrated IC50 similar to free drug.

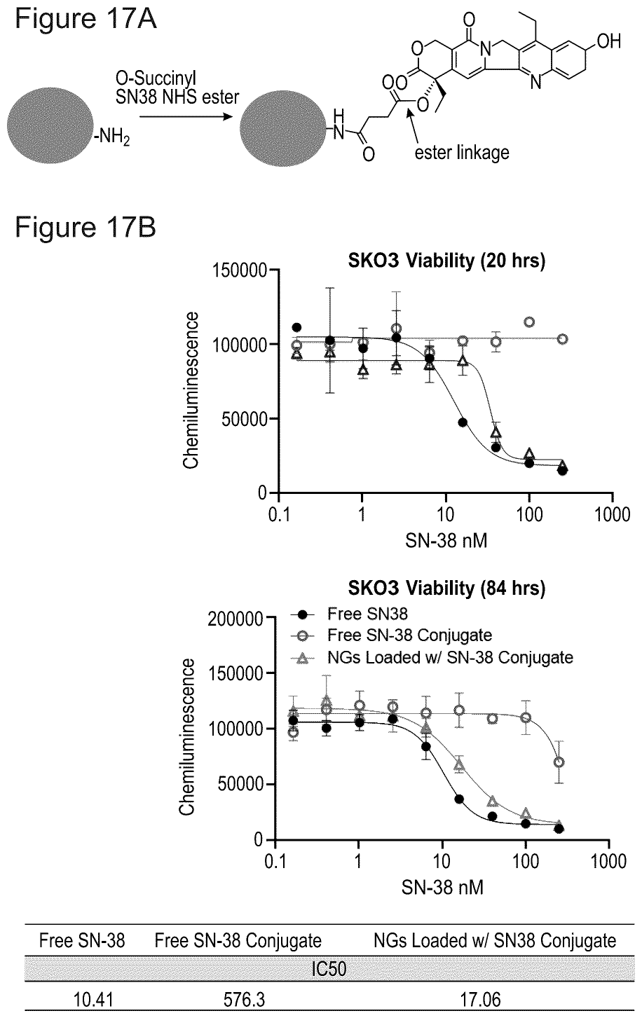

As another strategy to increase SN-38 solubilization, SN-38 is conjugated to a protein (albumin) or a peptide via different linkers (for stability and specificity to intracellular enzymes) and then loaded into NGs. Initial studies used ester linker (FIG. 17A) to bind SN-38 to albumin. This conjugate was loaded during the downsizing step (with XStream high shear homogenizer) of NGs production, as it was found as the most efficient strategy for loading. The loading conditions were based on a set of optimization studies carried out with a model protein (AF488 fluorescently labeled BSA; data not shown). As shown in FIG. 17B, SN-38 conjugated to albumin (two SN-38 molecules per one albumin molecule, in an average), demonstrated IC50 that is similar to free SN-38.

### Example 11

### NGs Loading with IL-12 mRNA

IL-12 is a pro-inflammatory type 1 cytokine and a central mediator of the TH1 immune response leading to (among others) enhanced activation and cytotoxic activity of NK (natural killer cells), NKT (NK T cells) and CTLs (cytotoxic T lymphocytes), enhanced Ag presentation, and T cell recruitment. Early clinical trials (mid-1990's) showed that systemic delivery of IL-12 incurred dose-limiting toxicities. To minimize system toxicity, strategies to target IL-12 delivery to the tumor microenvironment are being developed, including IL-12 mRNA-based therapies that are delivered by local intra-tumoral injection (Nguyen et al., 2020 Front. Immunol., 2020, 11: Article 575597). Encouraging results were recently observed with Moderna's IL-12 mRNA MEDI1191 in 2L+ advanced/metastatic solid tumor patients (Hamid et al., ESMO TAT Conference; NCT03946800), further supporting development of this payload.

Developing NGs loaded with IL-12 mRNA that will allow IV administration and targeted delivery to the tumor will overcome the risks and complexities related to intratumoral injection.

### Results

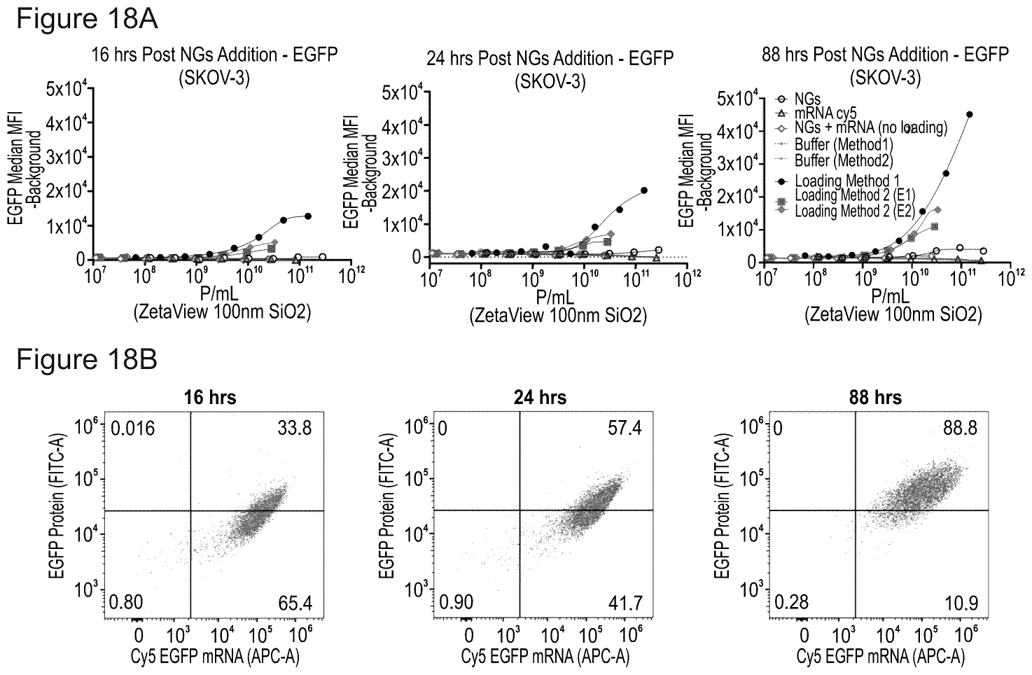

The present inventors used EGFP mRNA to optimize the loading conditions of mRNA within NGs. As shown in FIGS. 18A-B, Cy5 labeled EGFP mRNA was efficiently loaded during the downsizing step (with XStream high shear homogenizer) of 0.65 filtered particles to NGs. Following uptake of mRNA loaded NGs to cancer cells, EGFP expression was observed for at least 88 hrs in SKOV-3 cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of producing spherical particles, the method comprising:
(a) subjecting cells to a hypotonic treatment so as to obtain swollen intact cells;
(b) subjecting said swollen intact cells to flow shearing to obtain ruptured cells while avoiding nuclei lysis;
(c) filtering said ruptured cells to obtain a cell preparation devoid of nuclei;
(d) subjecting said cell preparation devoid of nuclei to size separation to obtain ghosts;
(e) downsizing said ghosts using a high shear homogenizer or microfluidizer, to obtain particles of 35-400 nm; and optionally
(f) purifying said particles to obtain said spherical particles.

2. The method of claim 1, wherein said cells are mesenchymal stem cells (MSCs), optionally wherein said MSCs having been cultured in suspension, optionally wherein said cells are provided at an amount of at least 0.5×10⁹, optionally wherein said MSCs exhibit a population doubling level (PDL) of 15-30.

3-5. (canceled)

6. The method of claim 1, further comprises culturing said cells prior to step (a), optionally wherein said culturing is in a bioreactor.

7-8. (canceled)

9. The method of claim 1, wherein said hypotonic treatment is under dynamic conditions, or wherein osmolarity of said hypotonic treatment is 5-100 mOsm/Kg or wherein said hypotonic treatment is effected for 10-60 minutes or wherein said hypotonic treatment results in cell swelling by at least 20% as determined by cell diameter.

10-12. (canceled)

13. The method of claim 1, wherein said flow shearing is performed by a needle, tube or channel optionally said needle/tube/channel being characterized by an internal diameter (ID) of 100-400 μm and/or a needle/tube/channel length of 2-50 mm.

14. The method of claim 1, wherein said flow shearing is performed with a single or multi-needle/tube/channel apparatus.

15. (canceled)

16. The method of claim 1, wherein said filtering said ruptured cells to obtain a cell preparation devoid of nuclei comprises a first filtration at a filter cut-off of 1.2-10 μm to remove nuclei, and optionally a second filtration at a filter cut-off of 0.45-0.85 μm to remove intracellular organelles.

17. The method of claim 1, wherein said size separation is performed by a size exclusion column.

18. The method of claim 1, wherein said size separation is performed by Tangential Flow Filtration (TFF).

19. The method of claim 1, wherein operating conditions for said high shear homogenizer or microfluidizer include at least 100 bar.

20. The method of claim 1, wherein said high shear homogenizer is of 100-2000 bar.

21. The method of claim 1, further comprising subjecting said ghosts to size separation following said downsizing.

22. The method of claim 1, wherein said purifying comprises filtering said particles to obtain spherical particles at a filter cut-off of 0.2-0.22 μm.

23. A composition comprising a plurality of spherical particles composed of a whole cell membrane fraction, wherein the spherical particles exhibit native membrane symmetry and expression of native markers obtainable according to the method of claim 1.

24. The composition of claim 23, wherein the composition is characterized by a membrane to nucleus marker ratio higher than that obtained when using a homogenizer instead of said flow shearing to rupture cells or when collecting the centrifugal pellet following homogenization instead of the filtrate following the flow shearing.

25. A method of producing a pharmaceutical composition, the method comprising producing the composition of claim 24 and adding a pharmaceutical agent of interest following said size separation and prior to said downsizing so as to obtain spherical particles encapsulating said pharmaceutical agent.

26. A composition comprising a plurality of spherical particles of claim 23 wherein said spherical particles encapsulate a pharmaceutical agent.

27-30. (canceled)

31. The composition of claim 26, wherein said pharmaceutical agent comprises an mRNA or a water insoluble drug, optionally wherein said water insoluble drug comprises irinotecan or its active derivative SN-38, optionally wherein said water insoluble drug is in a formulation for increasing water solubility, optionally wherein said water insoluble drug is attached to a cyclodextrin, a protein or a peptide.

32-34. (canceled)
